(19) United States
(12) Patent Application Publication
(10) Pub. No.: US 2024/0389453 A1
(43) Pub. Date: Nov. 21, 2024

(54) ORGANIC ELECTROLUMINESCENT DIODE AND DISPLAY PANEL

(71) Applicant: BOE Technology Group Co., Ltd., Beijing (CN)

(72) Inventors: Haiyan SUN, Beijing (CN); Xiaojin ZHANG, Beijing (CN); Lixia QIU, Beijing (CN); Dan WANG, Beijing (CN)

(73) Assignee: BOE Technology Group Co., Ltd., Beijing (CN)

(21) Appl. No.: 18/271,050

(22) PCT Filed: Apr. 28, 2022
(86) PCT No.: PCT/CN2022/090034

§ 371 Date: Jul. 6, 2023

(51) Int. Cl. H10K 85/60; C09K 11/06; H10K 50/11; H10K 50/16; H10K 101/40; H10K 102/00

(52) U.S. Cl. CPC H10K 85/6572; C09K 11/06; H10K 85/615; H10K 85/657; H10K 85/6574; C09K 2211/1018; H10K 50/11; H10K 50/16; H10K 2101/40; H10K 2102/351

(57) ABSTRACT

The present disclosure provides an organic electroluminescent diode, and belongs to the field of display technology. The organic electroluminescent diode includes an anode, a light emitting layer, a hole blocking layer, an electron transport layer, and a cathode, which are stacked in sequence; wherein the light emitting layer comprises a host material, a TADF material, and a fluorescent dopant material; the host material is selected from the compound represented by Chemical Formula 1, and the material of the hole blocking layer is selected from the compound represented by Chemical Formula 2: The organic electroluminescence diode can improve the lifetime of the diode.

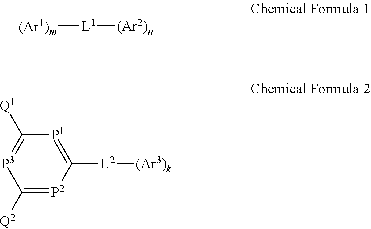

### TECHNICAL FIELD

This disclosure relates to the field of display technology, and particularly to an organic electroluminescent diode and a display panel.

### BACKGROUND

The superfluorescence technology based on TADF (thermal activation delayed fluorescence) sensitizer is considered one of the most valuable OLED (organic electroluminescent diode) technologies. However, superfluorescent OLEDs typically face issues of rapid device degradation and low lifetime.

It should be noted that the information disclosed in the background art section above is only used to enhance the understanding of the background of the disclosure, and therefore may include information that does not constitute the prior art known to those of ordinary skill in the art.

### SUMMARY OF THE INVENTION

The purpose of the disclosure is to overcome the above-mentioned shortcomings of the prior art, and provide an organic electroluminescent diode and a display device to improve the lifetime of the organic electroluminescent diode.

According to an aspect of the disclosure, an organic electroluminescent diode is provided, including an anode, a light emitting layer, a hole blocking layer, an electron transport layer, and a cathode that are stacked in sequence. The light emitting layer includes a host material, a TADF material, and a fluorescent dopant material.

The host material is selected from the compound represented by Chemical Formula 1, and the material of the hole blocking layer is selected from the compound represented by Chemical Formula 2:

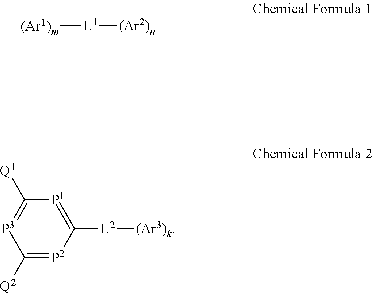

Wherein, m and n are the same or different, and are independently selected from an integer not less than 1; K is 1 or 2.

L¹ is selected from a single bond, and substituted or unsubstituted aryl group with 6 to 12 ring-forming carbon atoms. In a case that L¹ has a substituent, the substituent is selected from deuterium, fluorine, cyano, an alkyl group with 1 to 4 carbon atoms, deuteroalkyl group with 1 to 4 carbon atoms, fluoroalkyl group with 1 to 4 carbon atoms, and aryl group with 6 to 12 ring-forming carbon atoms.

Ar¹ is selected from the groups shown in Chemical Formulas 1-A, 1-B, and 1-C:

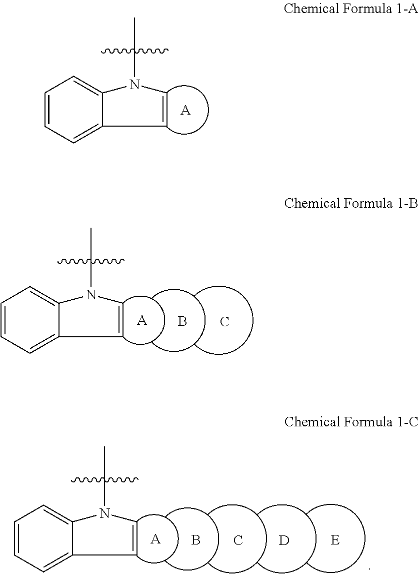

Ring A, Ring C, and Ring E are independently substituted or unsubstituted benzene ring. In a case that Ring A, Ring C, or Ring E has a substituent, the substituent is selected from deuterium, fluorine, cyano, alkyl group with 1 to 4 carbon atoms, deuteroalkyl group with 1 to 4 carbon atoms, and fluoroalkyl group with 1 to 4 carbon atoms.

Ring B is

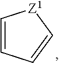

### and Ring D is

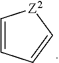

Z¹ and Z² are independently selected from NR¹, O, S, C(R²R³), Si(R²R³), and Ge(R²R³). Wherein, R¹, R², and R³ are the same or different, and each independently selected from hydrogen, alkyl group with 1 to 4 carbon atoms, and aryl group with 6 to 12 ring-forming carbon atoms.

Ar² is selected from the groups shown in Chemical Formulas 1-D, 1-E, 1-F, and 1-G:

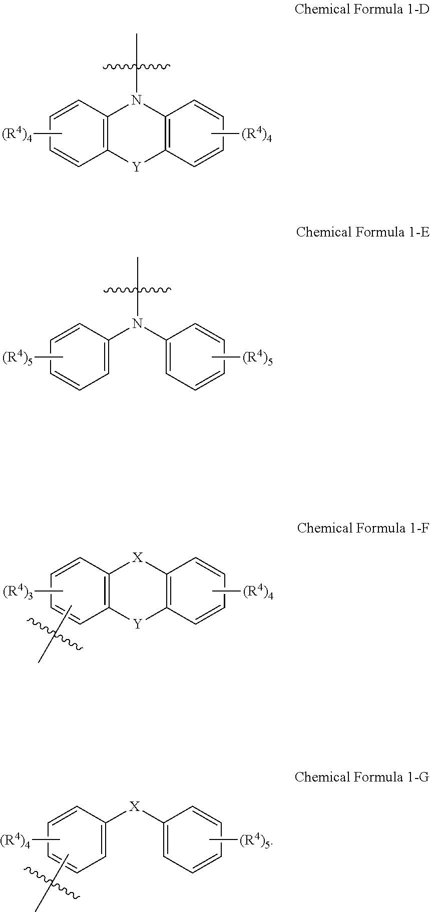

Each R⁴ is the same or different, and is independently selected from hydrogen, deuterium, fluorine, cyano, alkyl group with 1 to 4 carbon atoms, deuteroalkyl group with 1 to 4 carbon atoms, fluoroalkyl group with 1 to 4 carbon atoms, aryl group with 6 to 12 ring-forming carbon atoms, and heteroaryl group with 3 to 15 ring-forming carbon atoms.

X and Y are independently selected from NR⁵, O, S, C(R⁶R⁷), Si(R⁶R⁷), Ge(R⁶R⁷). R⁵, R⁶, and R⁷ are independently selected from hydrogen, alkyl group with 1 to 4 carbon atoms, and aryl group with 6 to 12 ring-forming carbon atoms, and Y can also be a single bond.

And Ar¹ and Ar² are not simultaneously N-carbazolyl group.

L² is selected from a single bond, substituted or unsubstituted aryl group with 6 to 12 ring-forming carbon atoms. In a case that the L² has a substituent, the substituent is selected from deuterium, fluorine, cyano, alkyl group with 1 to 4 carbon atoms, deuteroalkyl group with 1 to 4 carbon atoms, and fluoroalkyl group with 1 to 4 carbon atoms.

P¹, P², and P³ are the same or different, each independently selected from N or CH, and at least two are N.

Ar³ is selected from the groups shown in Chemical Formulas 2-A and 2-B:

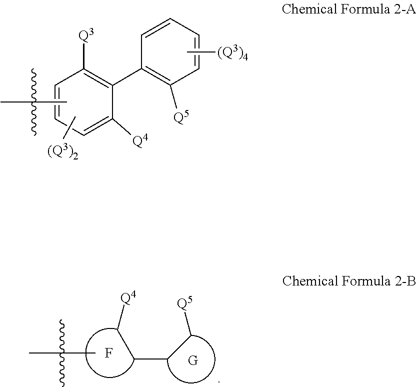

Ring F and Ring G are independently selected from benzene ring or pyridine ring, and at least one is pyridine ring.

Q¹, Q², and each Q³, Q⁴, and Q⁵ are the same or different, and are independently selected from hydrogen, deuterium, cyano, fluorine, substituted or unsubstituted aryl group with 5 to 50 carbon atoms, and substituted or unsubstituted alkyl group with 1 to 50 carbon atoms. Alternatively, Q⁴ and Q⁵ can be condensed to form 5- to 7-membered cycle with connected groups.

According to another aspect of the disclosure, a display panel is provided, including the aforementioned organic light emitting diode.

It should be understood that the above general description and the following detailed description are only exemplary and explanatory, and cannot limit the disclosure.

### BRIEF DESCRIPTION OF THE DRAWINGS

The drawings herein are incorporated into the specification and constitute a part of the specification, show embodiments in accordance with the disclosure, and are used together with the specification to explain the principle of the disclosure. Obviously, the drawings in the following description are only some embodiments of the disclosure. For those of ordinary skill in the art, other drawings can be obtained based on these drawings without creative work.

FIG. 1 is a schematic structural diagram of an organic electroluminescent diode in an embodiment of the disclosure.

FIG. 2 is a schematic structural diagram of a display panel in an embodiment of the disclosure an embodiment of the disclosure.

FIG. 3 is a schematic structural diagram of a display panel in an embodiment of the disclosure an embodiment of the disclosure.

FIG. 4 is a schematic structural diagram of multiple the organic electroluminescent diodes in the display panel in an embodiment of the disclosure.

### DETAILED DESCRIPTION

Example embodiments will now be described more fully with reference to the accompanying drawings. However, the example embodiments can be implemented in various forms, and should not be construed as being limited to the embodiments set forth herein. On the contrary, these embodiments are provided so that the disclosure will be comprehensive and complete, and fully convey the concept of the example embodiments to those skilled in the art. The same reference numerals in the figures indicate the same or similar structures, and thus their detailed descriptions will be omitted. In addition, the drawings are only schematic illustrations of the disclosure, and are not necessarily drawn to scale.

In the embodiment of the disclosure, the term “substituted or unsubstituted” refers to the functional groups defined by the term that may or may not have a substituent (hereinafter, the substituent is collectively referred to as Rc for ease of description). For example, “substituted or unsubstituted aryl group” refers to an aryl group with a substituent Rc or an unsubstituted aryl group. The substituent mentioned above, namely Rc, can be deuterium, halogen group, cyano, alkyl group, alkoxy group, alkylthio group, haloalkyl group, deuteroalkyl group, cycloalkyl group, trialkylsilyl group, triphenylsilyl group, diarylphosphinyl group, aryloxy group, and other groups. In the embodiment of the disclosure, the “substituted” functional group can be substituted with one or more substituents Rc as described above.

In the embodiment of the disclosure, the number of carbon atoms of a substituent or an unsubstituent refers to the number of carbon atoms in total. For example, if Ar¹ is a substituted aryl group with a carbon atom number of 12, then the total number of carbon atoms of the aryl group and the substituent(s) thereon combined are 12.

In the embodiment of the disclosure, the description mean of “each . . . independently” can refer to that the specific options shown by the same symbol in different groups do not affect each other, or that the specific options shown by the same symbol in the same group do not affect each other. For example, the description of

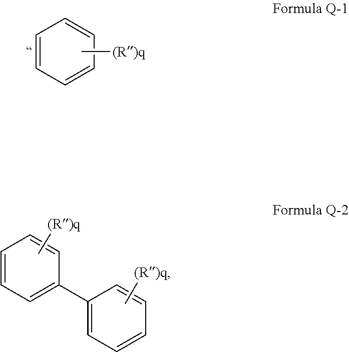

wherein each q is independently 0, 1, 2, 3, and each R “is independently selected from hydrogen, fluorine, or chlorine” means that Formula Q-1 indicates q substituent(s) R″ are combined on the benzene ring, and each R “can be the same or different, and the options of each R” do not affect each other; Formula Q-2 indicates that q substituent(s) R″ are combined on each benzene ring of biphenyl, the number q of substituent(s) R″ on both benzene rings can be the same or different, each R″ can be the same or different, and the options of each R″ do not affect each other.

In the embodiment of the disclosure, aryl group refers to an optional functional group or substituent derived from an aromatic hydrocarbon ring. Aryl group can be monocyclic aryl group (e.g. phenyl) or polycyclic aryl group. In other words, aryl group can be monocyclic aryl group, fused aryl group, two or more monocyclic aryls conjugated by carbon-carbon bond, monocyclic aryl group and fused aryl group conjugated by carbon-carbon bond, and two or more fused aryls conjugated by carbon-carbon bond. That is, two or more aromatic groups conjugated by carbon-carbon bond can also be considered as aryl in the embodiment of the disclosure. Wherein, fused aryl group can include, for example, bicyclic fused aryl group (e.g. naphthyl), tricyclic fused aryl group (e.g. phenyl, fluorenyl, anthracyl), and the like. Aryl group does not contain heteroatoms such as B, N, O, S, Se, Si, or P. For example, in the embodiment of the disclosure, biphenyl, triphenyl and the like are aryl groups. Examples of aryl group may include, but are not limited to, phenyl, naphthyl, fluorenyl, anthryl, phenanthryl, biphenyl, triphenyl, quaterphenyl, benzo[9,10] phenyl, pyrenyl, benzofluoranthyl, cherysenyl, and the like.

In the embodiment of the disclosure, the substituted aryl group can aryl in which one or two or more hydrogen atoms are substituted with group such as deuterium atom, halogen group, —CN, aryl group, heteroaryl group, trialkylsilyl group, alkyl group, cycloalkyl group, alkoxy group, alkylthio group, and the like. The specific example of heteroaryl substituted aryl group includes, but is not limited to, dibenzofuranyl-substituted phenyl group, dibenzothiophenyl-substituted phenyl group, pyridyl-substituted phenyl group, carbazolyl-substituted phenyl group, and the like. It should be understood that the carbon atom number of the substituted aryl group refers to the total number of carbon atoms of the aryl group and the substituent(s) thereon combined. For example, for a substituted aryl group with a carbon atom number of 18, it means that the total carbon atom number of the aryl group and the substituent(s) combined are 18.

In the embodiment of the disclosure, heteroaryl group refers to a monovalent aromatic ring or its derivative, and the heteroatom can be at least one of B, O, N, P, Si, Se and S. The heteroaryl group can be monocyclic heteroaryl group or polycyclic heteroaryl group, in other words, the heteroaryl group can be a single aromatic ring system, and can also be multiple aromatic ring systems conjugatedly connected by a carbon-carbon bond, where any one of the aromatic ring system is an aromatic monocyclic ring or an aromatic fused ring. For example, the heteroaryl group can include, but be not limited to, thienyl group, furyl group, pyrrolyl group, imidazolyl group, thiazolyl group, oxazolyl group, oxadiazolyl group, triazolyl group, pyridyl group, bipyridyl group, pyrimidinyl group, triazinyl group, acridinyl group, pyridazinyl group, pyrazinyl group, quinolyl group, quinazolinyl group, quinoxalinyl group, phenoxazinyl group, phthalazinyl group, pyridopyrimidinyl group, pyridopyrazinyl, pyrazinopyrazinyl group, isoquinolyl group, indolyl group, carbazolyl group, benzoxazolyl group, benzoimidazolyl group, benzothiazolyl group, benzocarbazolyl group, benzothienyl group, dibenzothienyl group, thienothienyl, benzofuryl group, phenanthrolinyl group, isoxazolyl group, thiadiazoly group, benzothiazolyl group, Phenothiazinyl group, silafluorenyl group, dibenzofuranyl group, N-arylcarbazolyl group (e.g. N-phenylcarbazolyl group), N-heteroarylcarbazolyl group (e.g. N-pyridylcarbazolyl group), N-alkylcarbazolyl group (e.g. N-methylcarbazolyl group), and the like. Wherein, thienyl group, furyl group, phenanthrolinyl group, or the like is heteroaryl group of single aromatic ring system type, and N-arylcarbazolyl group or N-heteroarylcarbazolyl group is heteroaryl group of polycyclic system type conjugated by carbon-carbon bond.

In the embodiment of the disclosure, the substituted heteroaryl group can be aryl in which one or two or more hydrogen atoms are substituted with a group such as deuterium atom, halogen group, —CN, aryl group, heteroaryl group, trialkylsilyl group, alkyl group, cycloalkyl group, alkoxy group, alkylthio group, and the like. Specific examples of aryl substituted heteroaryl groups include, but are not limited to, phenyl-substituted dibenzofuranyl group, phenyl-substituted dibenzothiophenyl group, phenyl-substituted pyridinyl group, and the like. It should be understood that the number of carbon atoms in the substituted heteroaryl group refers to the total number of carbon atoms in the heteroaryl group and the substituents thereon combined.

In the embodiment of the disclosure, the unpositioned connecting bond refers to a single bond “ [symbol] ” extending from the ring system, which means that one end of the connecting bond can be connected to any position of the ring system through which the bond passes, and the other end can be connected to the rest of the compound molecule.

For example, as shown in Formula (f) below, the naphthyl group represented by Formula (f) is connected to other positions of the molecule through two unpositioned connecting bonds which pass through a dicyclic ring. It means that it can include any possible connection way as shown in Formulas (f-1) to (f-10).

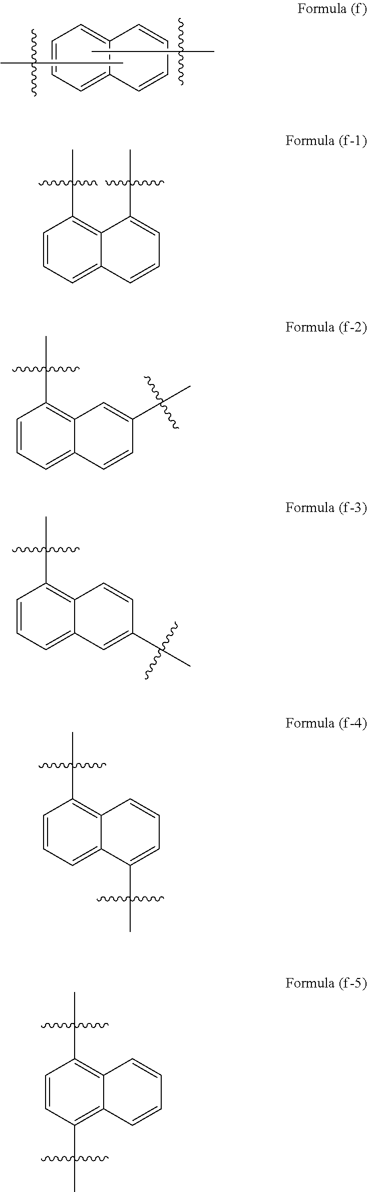

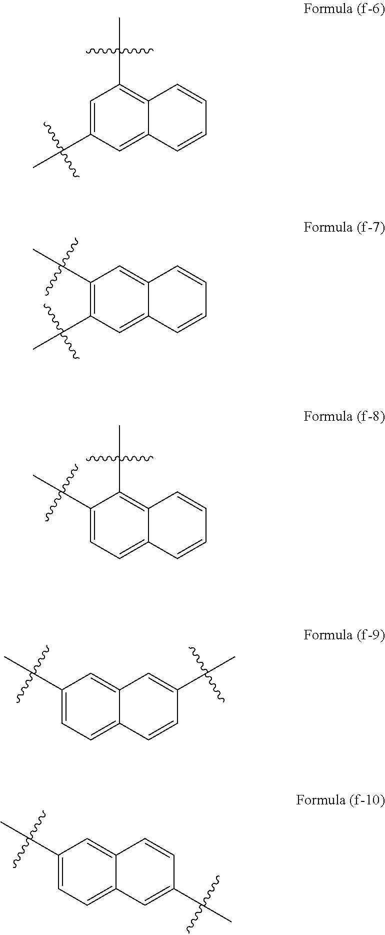 [SMILES: *c1cc(*)c2ccccc2c1 ; *c1cc2ccccc2cc1* ; *c1ccc2ccccc2c1* ; *c1ccc2ccc(*)cc2c1 ; *c1ccc2cc(*)ccc2c1]

For example again, as shown in Formula (X′) below, the phenyl group represented by Formula (X′) is connected to other positions of the molecule through an unpositioned connecting bond extending from one side of the benzene ring. It means that it can include any possible connection way as shown in Formulas (X′-1) to (X′-4).

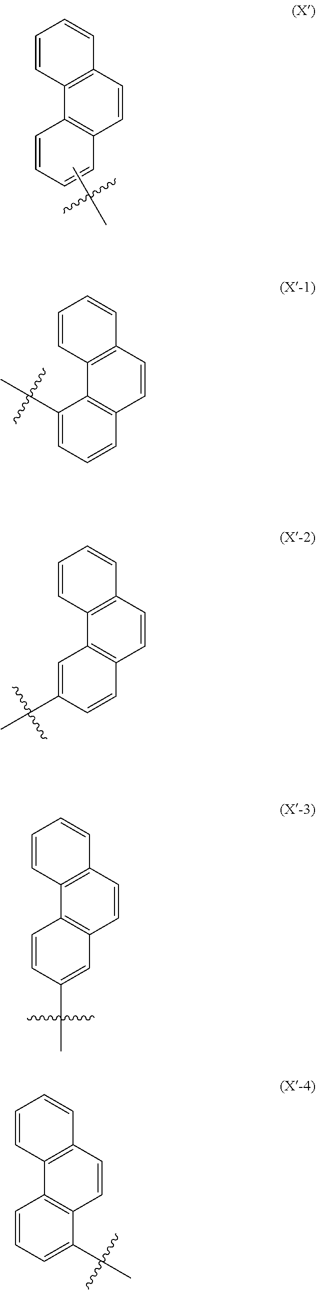

In the embodiment of the disclosure, the unpositioned substituent refers to a substituent connected by a single bond extending from the ring system, which means that the substituent can be connected at any possible position of the ring system. For example, as shown in Formula (Y) below, the substituent R′ group represented by Formula (Y) is connected to the quinoline ring through an unpositioned connecting bond. It means that it can include any possible connection way as shown in Formulas (Y-1) to (Y-7).

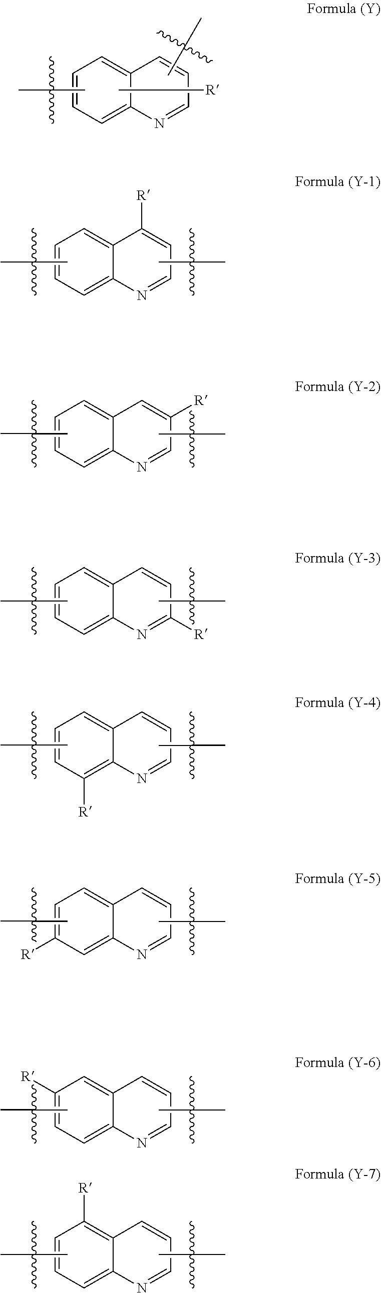

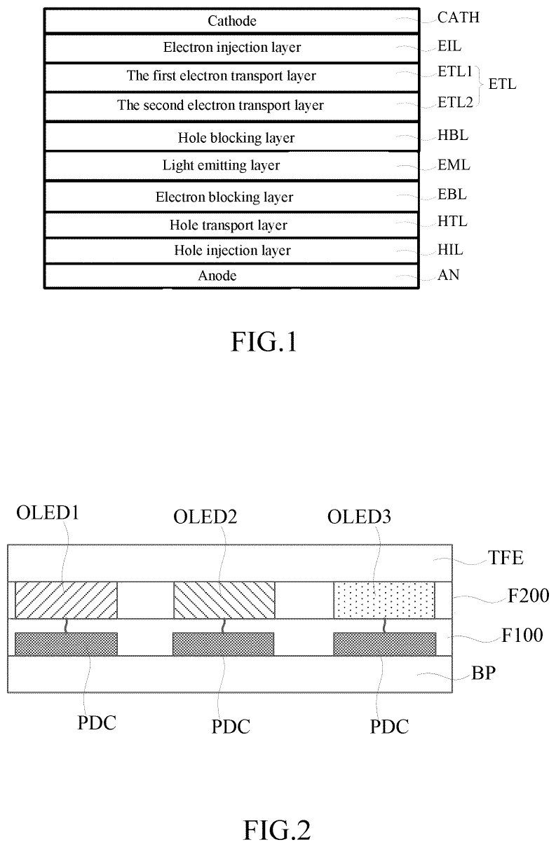

The embodiment of the disclosure provides an organic electroluminescent diode. Referring to FIG. 1, the organic electroluminescent diode includes an anode AN, a light emitting layer EML, a hole blocking layer HBL, an electron transport layer ETL, and a cathode CATH, which are stacked in sequence. The light emitting layer EML includes a host material, a TADF (thermal activation delayed fluorescence) material, and a fluorescent dopant material. The host material is used for transporting carriers, such as at least one of electron and hole. In the embodiment of the disclosure, the host material is a hole-biased type material, that is the material having the hole migration rate that is greater than the electron migration rate. By the transmission of the host material, the electrons and holes injected into the light emitting layer are mainly recombinated on the TADF material (as an assistant material), which transfers the energy (excitons) generated by the recombination to the fluorescent dopant material, causing the fluorescent dopant material to emit fluorescent light.

In the embodiment of the disclosure, the host material is selected from the compound represented by Chemical Formula 1, and the material of the hole blocking layer is selected from the compound represented by Chemical Formula 2:

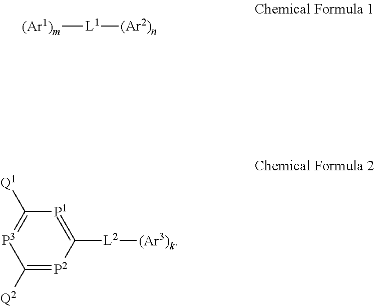

Wherein, m and n are the same or different, and are independently selected from an integer not less than 1; K is 1 or 2.

L¹ is selected from a single bond, and substituted or unsubstituted aryl group with 6 to 12 ring-forming carbon atoms. In a case that L¹ has a substituent, the substituent is selected from deuterium, fluorine, cyano, alkyl group with 1 to 4 carbon atoms, deuteroalkyl group with 1 to 4 carbon atoms, fluoroalkyl group with 1 to 4 carbon atoms, and aryl group with 6 to 12 ring-forming carbon atoms.

Ar¹ is selected from the groups shown in Chemical Formulas 1-A, 1-B, and 1-C:

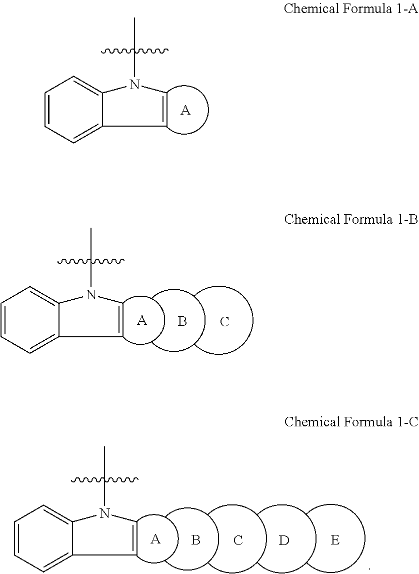

Ring A, Ring C, and Ring E are independently substituted or unsubstituted benzene ring. In a case that Ring A, Ring C, or Ring E has a substituent, the substituent is selected from deuterium, fluorine, cyano, alkyl group with 1 to 4 carbon atoms, deuteroalkyl group with 1 to 4 carbon atoms, and fluoroalkyl group with 1 to 4 carbon atoms.

Ring B is

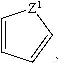

### and Ring D is

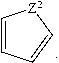

Z¹ and Z² are independently selected from NR¹, O, S, C(R²R³), Si(R²R³), and Ge(R²R³). Wherein, R¹, R², and R³ are the same or different, and each independently selected from hydrogen, alkyl group with 1 to 4 carbon atoms, and aryl group with 6 to 12 ring-forming carbon atoms.

Ar² is selected from the groups shown in Chemical Formulas 1-D, 1-E, 1-F, and 1-G:

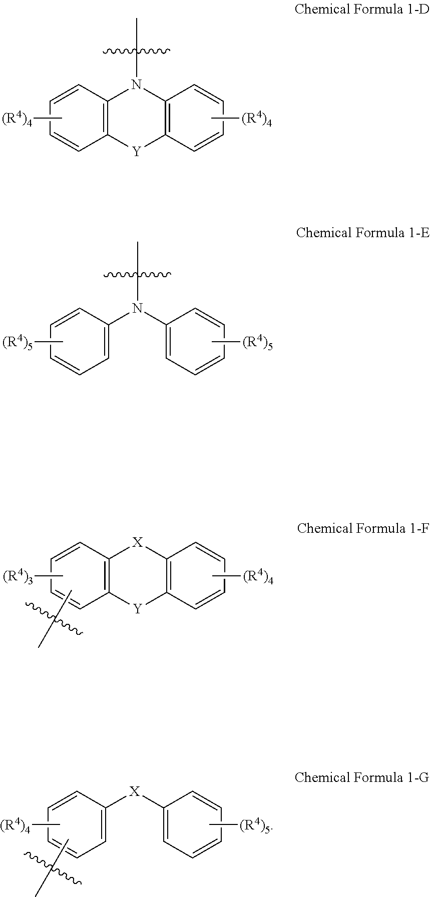

Each R⁴ is the same or different, and is independently selected from hydrogen, deuterium, fluorine, cyano, alkyl group with 1 to 4 carbon atoms, deuteroalkyl group with 1 to 4 carbon atoms, fluoroalkyl group with 1 to 4 carbon atoms, aryl group with 6 to 12 ring-forming carbon atoms, and heteroaryl group with 3 to 15 ring-forming carbon atoms.

X and Y are independently selected from NR⁵, O, S, C(R⁶R⁷), Si(R⁶R⁷), Ge(R⁶R⁷). R⁵, R⁶, and R⁷ are independently selected from hydrogen, alkyl group with 1 to 4 carbon atoms, and aryl group with 6 to 12 ring-forming carbon atoms, and Y can also be a single bond.

And Ar¹ and Ar² are not simultaneously N-carbazolyl group.

L² is selected from a single bond, substituted or unsubstituted aryl group with 6 to 12 ring-forming carbon atoms. In a case that the L² has a substituent, the substituent is selected from deuterium, fluorine, cyano, alkyl group with 1 to 4 carbon atoms, deuteroalkyl group with 1 to 4 carbon atoms, and fluoroalkyl group with 1 to 4 carbon atoms.

P¹, P², and P³ are the same or different, each independently selected from N or CH, and at least two are N.

Ar³ is selected from the groups shown in Chemical Formulas 2-A and 2-B:

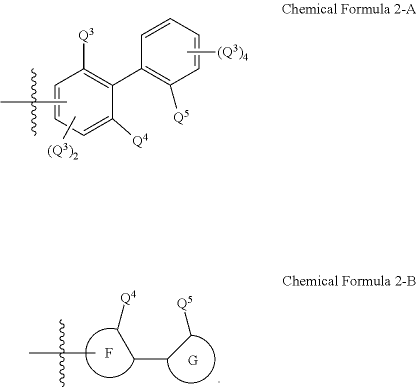

Ring F and Ring G are independently selected from benzene ring or pyridine ring, and at least one is pyridine ring.

Q¹, Q², and each Q³, Q⁴, and Q⁵ are the same or different, and are independently selected from hydrogen, deuterium, cyano, fluorine, substituted or unsubstituted aryl group with 5 to 50 carbon atoms, and substituted or unsubstituted alkyl group with 1 to 50 carbon atoms. Alternatively, Q⁴ and Q⁵ can be condensed to form 5-to 7-membered cycle with connected groups.

In an embodiment of the disclosure, the material of the electron transport layer is also selected from the compound represented by Chemical Formula 2.

In an embodiment of the disclosure, both m and n are 1.

In an embodiment of the disclosure, L¹ is selected from phenyl and biphenyl.

In an embodiment of the disclosure, Z¹ and Z² are each independently selected form NH, O, S or CH₂.

In an embodiment of the disclosure, Ring A, Ring C or Ring E has not substituent.

In an embodiment of the disclosure, the group represented by Chemical Formula 1-B is selected from the following groups:

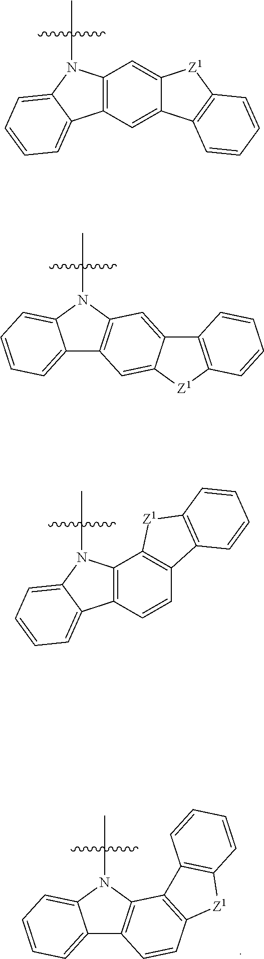

Chemical Formula 1-C is selected from the following groups:

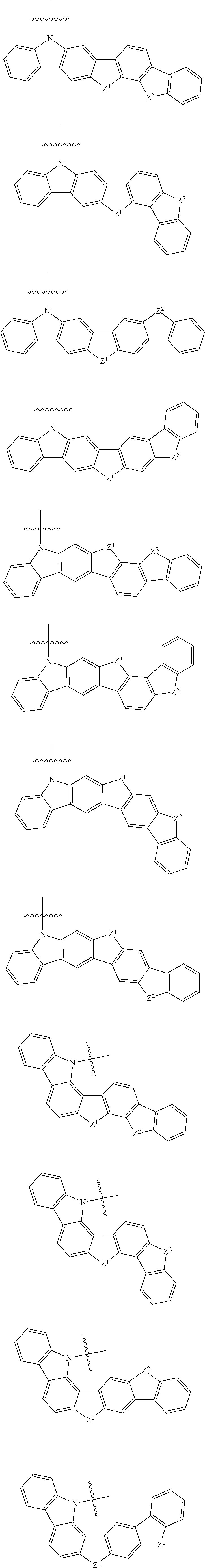

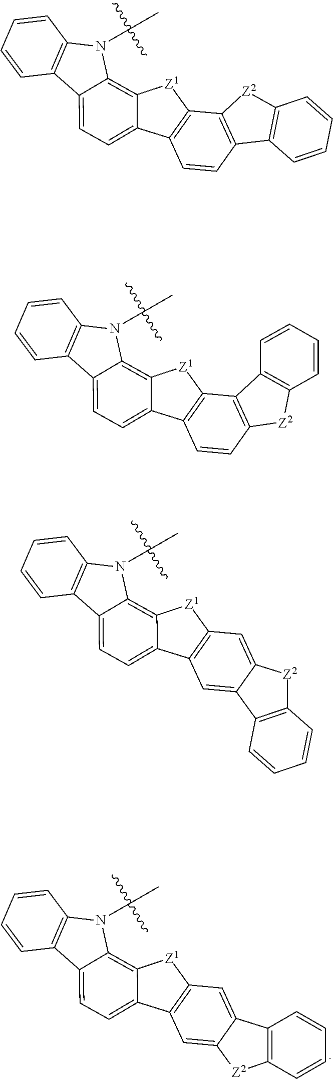

Ar¹ is selected from the following groups:

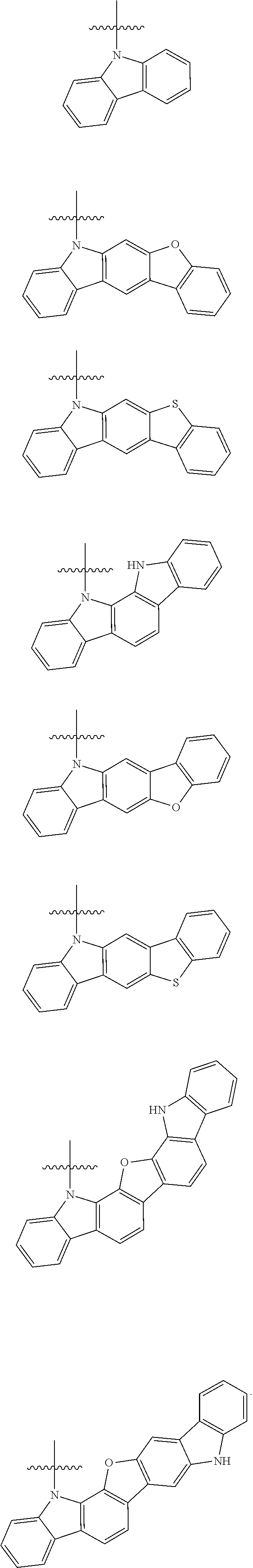

In an embodiment of the disclosure, R⁴ is hydrogen.

In an embodiment of the disclosure, X is not selected from NR⁵.

In an embodiment of the disclosure, X is selected from S, O, CMe₂, CPh₂.

In an embodiment of the disclosure, Y is selected from a single bond, S, O, CMe₂, CPh₂.

In an embodiment of the disclosure, R⁵, R⁶ and R⁷ are each independently selected from hydrogen, methyl and phenyl.

In an embodiment of the disclosure, Ar² is selected from the following groups:

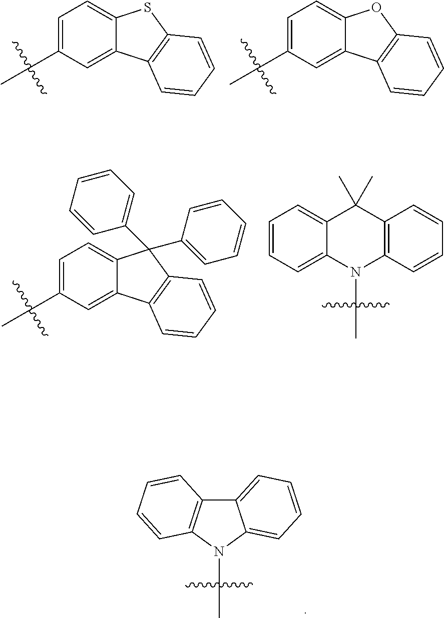

In an embodiment of the disclosure, the compound represented by Chemical Formula 1 is selected from the following groups:

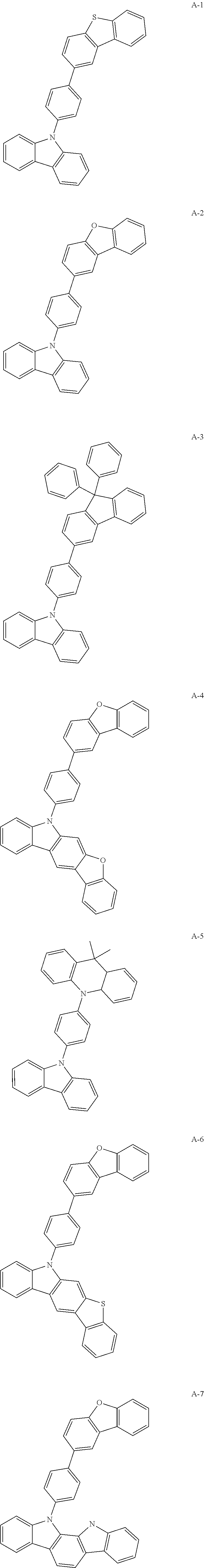

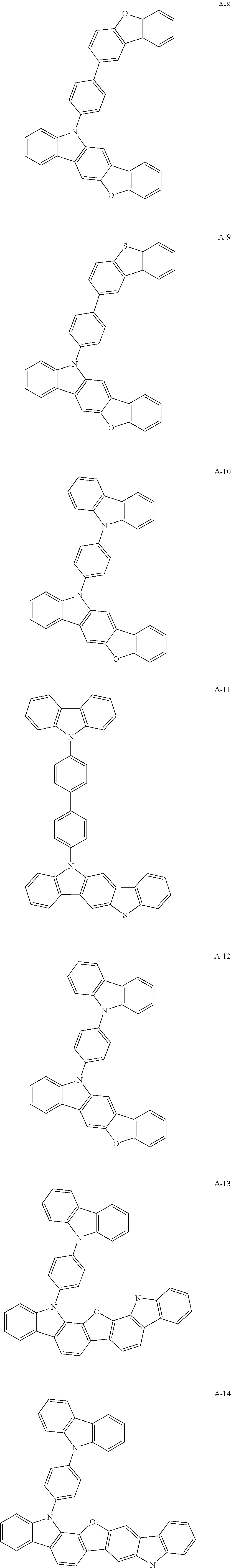

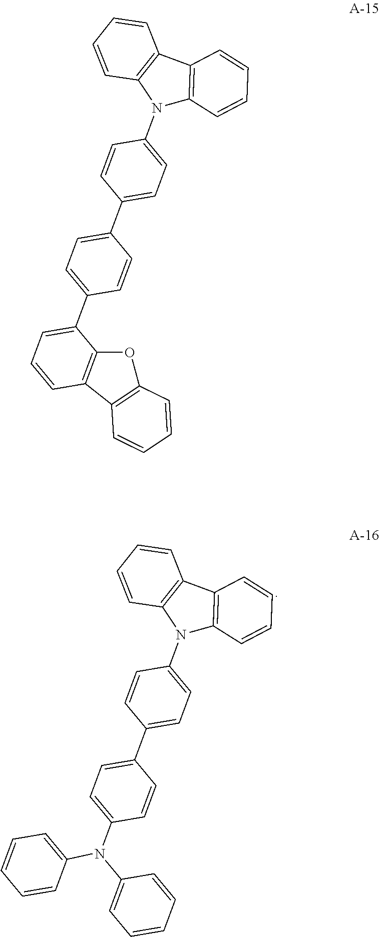 [SMILES: c1ccc2c(c1)oc1c(-c3ccc(-c4ccc(-n5c6ccccc6c6ccccc65)cc4)cc3)cccc12 ; c1ccc(N(c2ccccc2)c2ccc(-c3ccc(-n4c5ccccc5c5ccccc54)cc3)cc2)cc1]

In an embodiment of the disclosure, Q¹ and Q² are the same or different, and are each independently selected from hydrogen, deuterium, cyano, fluorine, aryl group with 6 to 12 ring-forming carbon atoms.

Furthermore, Q¹ and Q² are the same or different, and each is independently selected from phenyl and biphenyl.

In an embodiment of the disclosure, L² is selected from a single bond, phenyl, and biphenyl group.

In an embodiment of the disclosure, each Q³ is the same or different, and each Q³ is independently selected from hydrogen, deuterium, cyano, fluorine, aryl group with 6 to 12 ring-forming carbon atoms, and alkyl group with 1 to 4 carbon atoms. For example, it is selected from hydrogen, deuterium, phenyl, methyl, and the like, especially hydrogen.

In an embodiment of the disclosure, Q⁴ and Q⁵ are the same or different, and are independently selected from hydrogen, deuterium, cyano, fluorine, aryl group with 6 to 12 ring-forming carbon atoms, and alkyl group with 1 to 4 carbon atoms. For example, it is selected from hydrogen, deuterium, phenyl, methyl, and the like.

In an embodiment of the disclosure, Q⁴ and Q⁵ can be condensed to form 5- to 7-membered cycle with connected groups, such as furan ring, pyrrole ring, thiophene ring, cyclopentadiene ring, and the like. Wherein, the formed 5- to 7-membered cycle can be further substituted, for example, the methylene of cyclopentadiene ring can be substituted with two methyl groups or two phenyl groups, or the methylene of cyclopentadiene ring can be joined with diphenylfluorene in a spiro fashion.

In an embodiment of the disclosure, one of the Ring F and Ring G is a benzene ring, and the other is a pyridine ring.

In an embodiment of the disclosure, when Q⁴ and Q⁵ can be condensed to form 5- to 7-membered cycle with connected groups, the group represented by Chemical Formula 2-A is selected from the following groups:

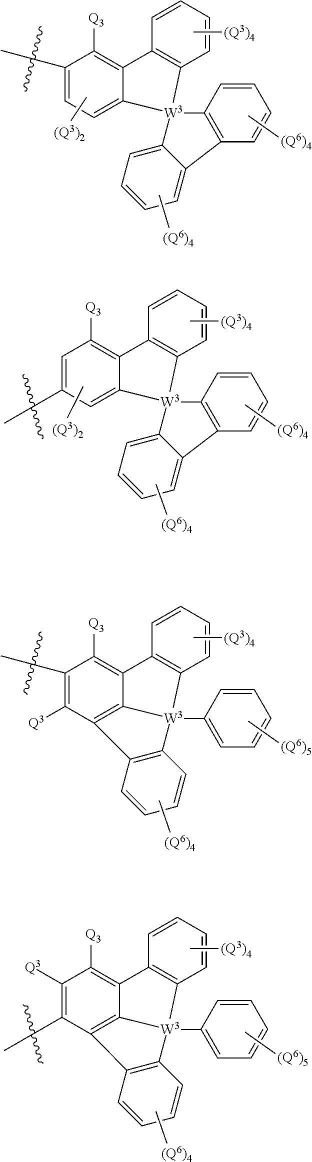

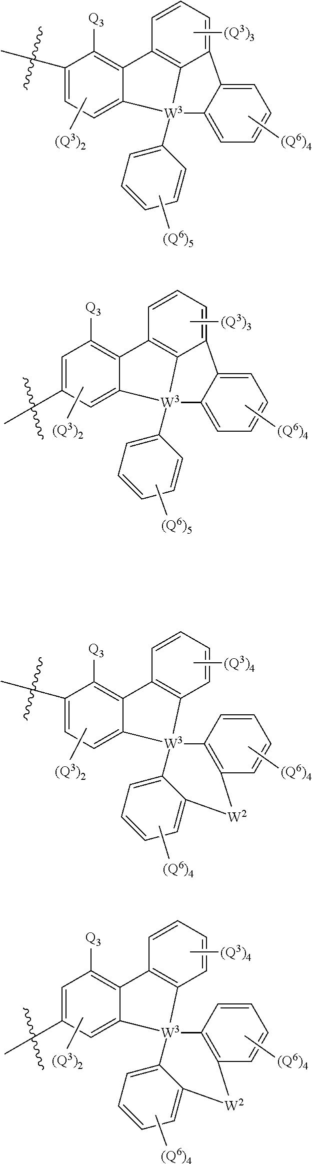

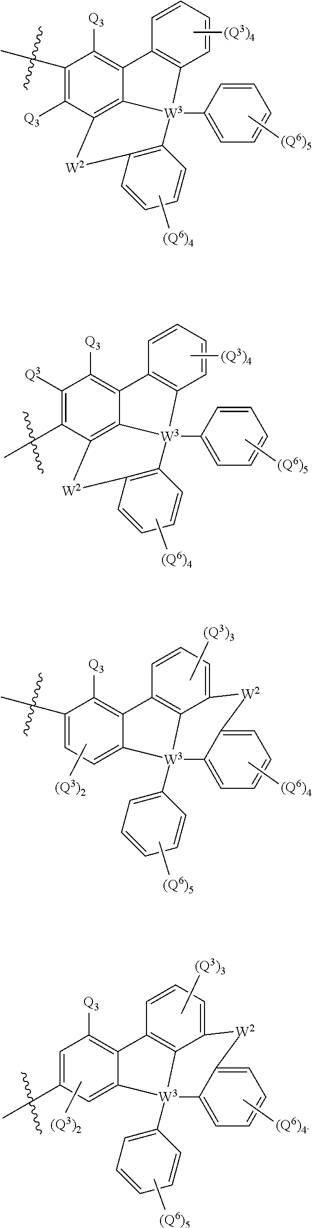

The group represented by Chemical Formula 2-B is selected from the following groups:

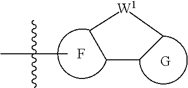

Wherein, W¹ is selected from NR⁸, O, and S, and R⁸ is selected from hydrogen, alkyl group with 1 to 4 carbon atoms, and aryl group with 6 to 12 ring-forming carbon atoms.

W² is selected from NR9 [symbol] O [symbol] S [symbol] C(R¹⁰R¹¹) [symbol] Si(R¹⁰R¹¹) [symbol] Ge(R¹⁰R¹¹). R⁹ is selected from hydrogen, alkyl group with 1 to 4 carbon atoms, and aryl group with 6 to 12 ring-forming carbon atoms. R¹⁰ and R¹¹ are independently selected from aryl group with 6 to 12 ring-forming carbon atoms.

W³ is selected from C, Si, and Ge.

Each Q⁶ is the same or different, and independently selected from hydrogen, deuterium, cyano, fluorine, substituted or unsubstituted aryl group with 5 to 50 ring-forming carbon atoms, and substituted or unsubstituted alkyl group with 1 to 50 carbon atoms.

In an embodiment of the disclosure, R⁸ is hydrogen.

In an embodiment of the disclosure, W³ is C.

In an embodiment of the disclosure, Ar³ is selected from the following groups:

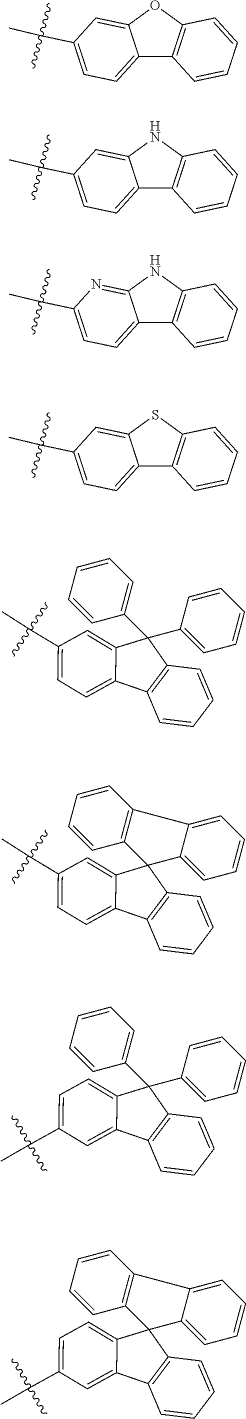

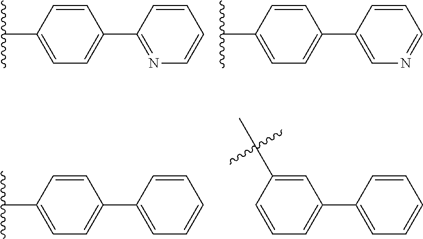

In an embodiment of the disclosure, the compound represented by Chemical Formula 2 is selected from the following groups:

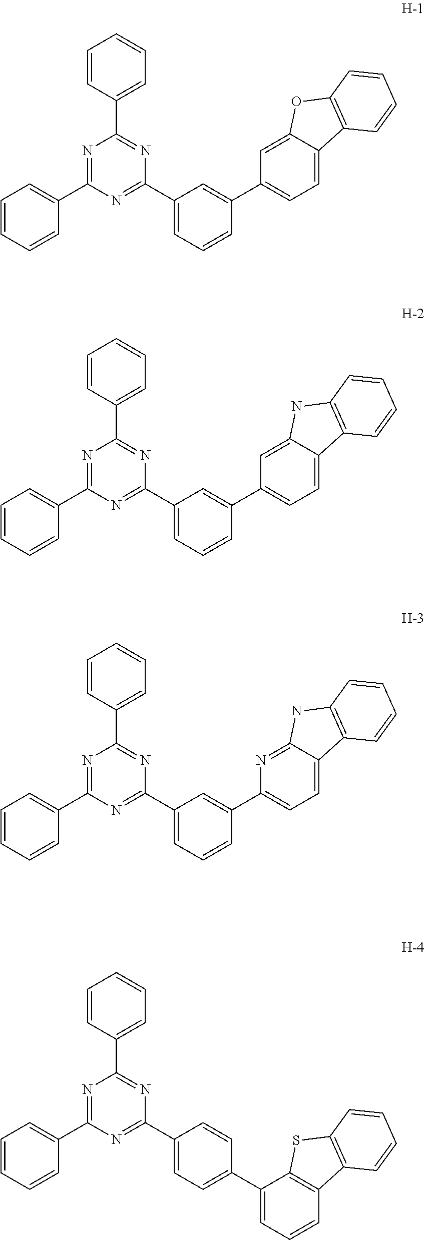 [SMILES: c1ccc(-c2nc(-c3ccccc3)nc(-c3cccc(-c4ccc5c(c4)oc4ccccc45)c3)n2)cc1 ; c1ccc(-c2nc(-c3ccccc3)nc(-c3cccc(-c4ccc5c(c4)[nH]c4ccccc45)c3)n2)cc1 ; c1ccc(-c2nc(-c3ccccc3)nc(-c3cccc(-c4ccc5c(n4)[nH]c4ccccc45)c3)n2)cc1 ; c1ccc(-c2nc(-c3ccccc3)nc(-c3ccc(-c4cccc5c4sc4ccccc45)cc3)n2)cc1]

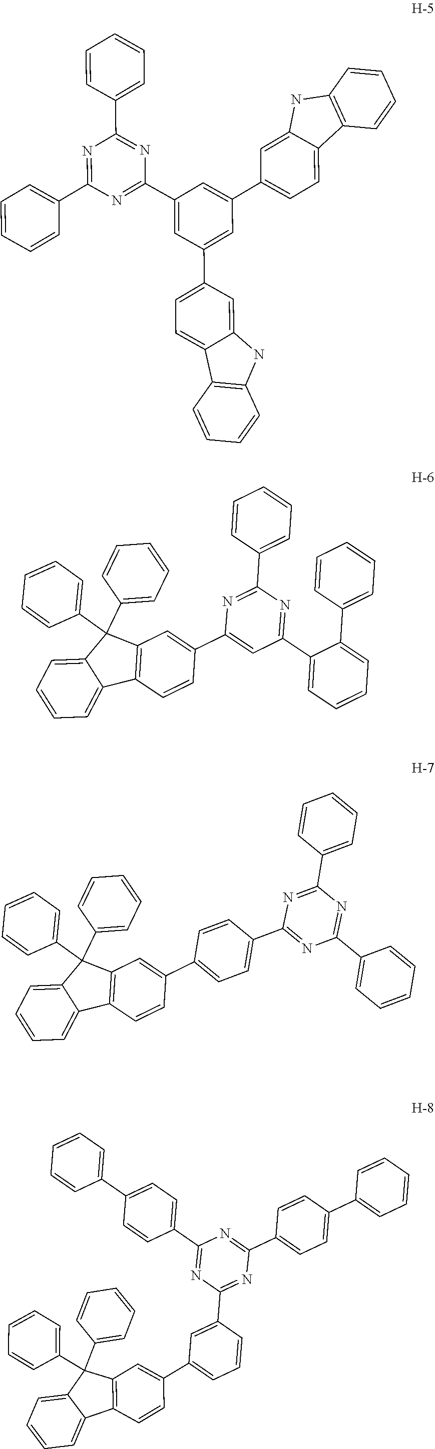

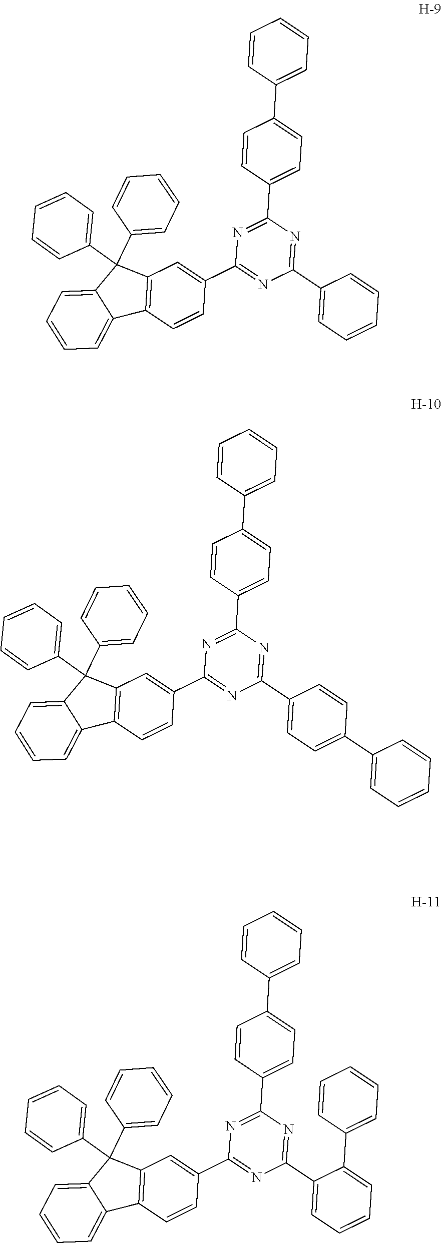 [SMILES: c1ccc(-c2ccc(-c3nc(-c4ccccc4)nc(-c4ccc5c(c4)C(c4ccccc4)(c4ccccc4)c4ccccc4-5)n3)cc2)cc1 ; c1ccc(-c2ccc(-c3nc(-c4ccc(-c5ccccc5)cc4)nc(-c4ccc5c(c4)C(c4ccccc4)(c4ccccc4)c4ccccc4-5)n3)cc2)cc1 ; c1ccc(-c2ccc(-c3nc(-c4ccc5c(c4)C(c4ccccc4)(c4ccccc4)c4ccccc4-5)nc(-c4ccccc4-c4ccccc4)n3)cc2)cc1]

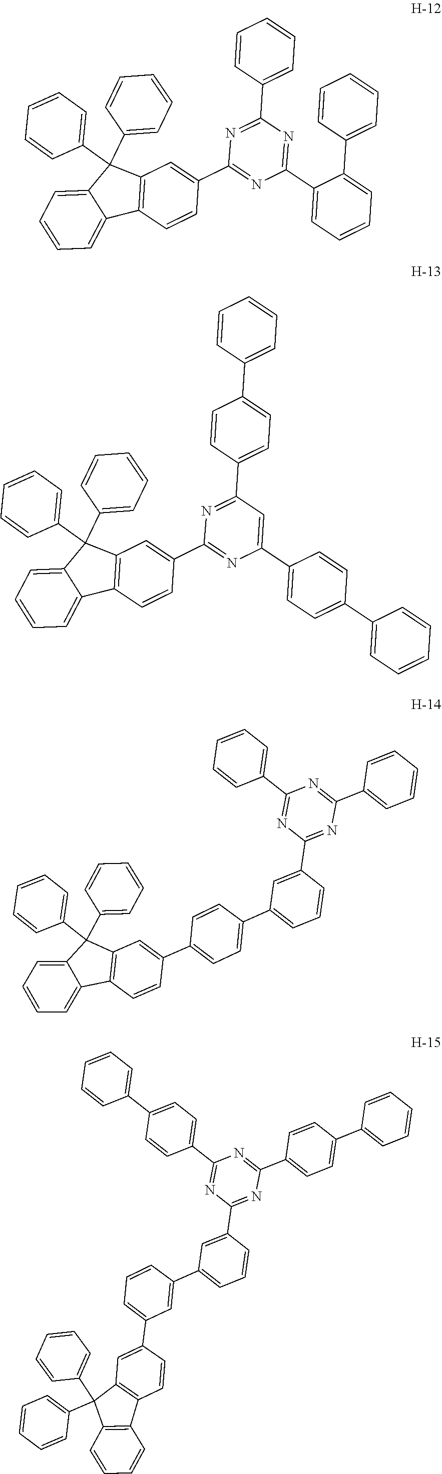 [SMILES: c1ccc(-c2nc(-c3ccc4c(c3)C(c3ccccc3)(c3ccccc3)c3ccccc3-4)nc(-c3ccccc3-c3ccccc3)n2)cc1 ; c1ccc(-c2ccc(-c3cc(-c4ccc(-c5ccccc5)cc4)nc(-c4ccc5c(c4)C(c4ccccc4)(c4ccccc4)c4ccccc4-5)n3)cc2)cc1 ; c1ccc(-c2nc(-c3ccccc3)nc(-c3cccc(-c4ccc(-c5ccc6c(c5)C(c5ccccc5)(c5ccccc5)c5ccccc5-6)cc4)c3)n2)cc1 ; c1ccc(-c2ccc(-c3nc(-c4ccc(-c5ccccc5)cc4)nc(-c4cccc(-c5cccc(-c6ccc7c(c6)C(c6ccccc6)(c6ccccc6)c6ccccc6-7)c5)c4)n3)cc2)cc1]

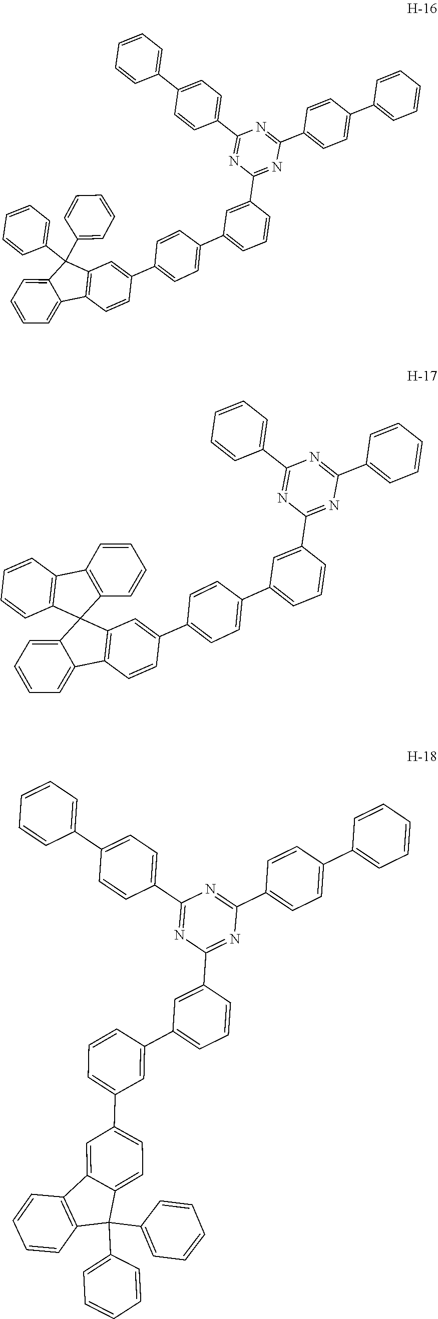 [SMILES: c1ccc(-c2ccc(-c3nc(-c4ccc(-c5ccccc5)cc4)nc(-c4cccc(-c5ccc(-c6ccc7c(c6)C(c6ccccc6)(c6ccccc6)c6ccccc6-7)cc5)c4)n3)cc2)cc1 ; c1ccc(-c2nc(-c3ccccc3)nc(-c3cccc(-c4ccc(-c5ccc6c(c5)C5(c7ccccc7-c7ccccc75)c5ccccc5-6)cc4)c3)n2)cc1 ; c1ccc(-c2ccc(-c3nc(-c4ccc(-c5ccccc5)cc4)nc(-c4cccc(-c5cccc(-c6ccc7c(c6)-c6ccccc6C7(c6ccccc6)c6ccccc6)c5)c4)n3)cc2)cc1]

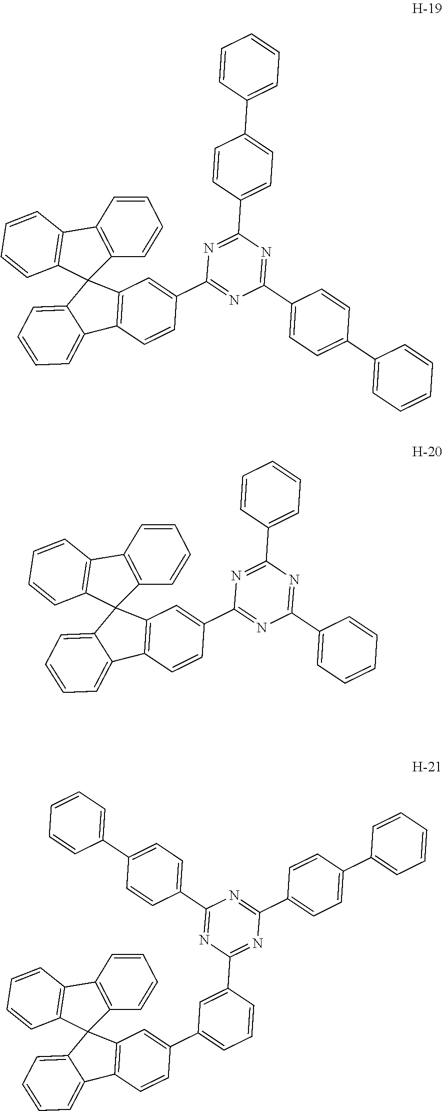 [SMILES: c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1 ; c1ccc(-c2nc(-c3ccccc3)nc(-c3ccc4c(c3)C3(c5ccccc5-c5ccccc53)c3ccccc3-4)n2)cc1 ; c1ccc(-c2ccc(-c3nc(-c4ccc(-c5ccccc5)cc4)nc(-c4cccc(-c5ccc6c(c5)C5(c7ccccc7-c7ccccc75)c5ccccc5-6)c4)n3)cc2)cc1]

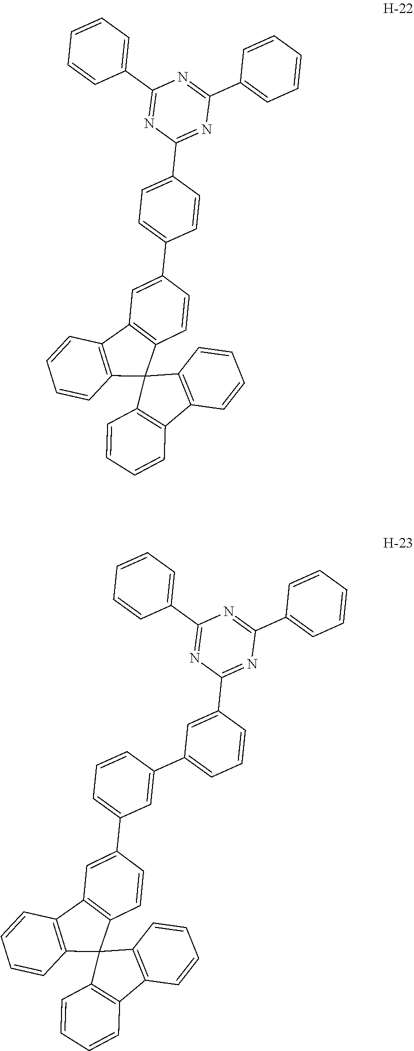 [SMILES: c1ccc(-c2nc(-c3ccccc3)nc(-c3ccc(-c4ccc5c(c4)-c4ccccc4C54c5ccccc5-c5ccccc54)cc3)n2)cc1 ; c1ccc(-c2nc(-c3ccccc3)nc(-c3cccc(-c4cccc(-c5ccc6c(c5)-c5ccccc5C65c6ccccc6-c6ccccc65)c4)c3)n2)cc1]

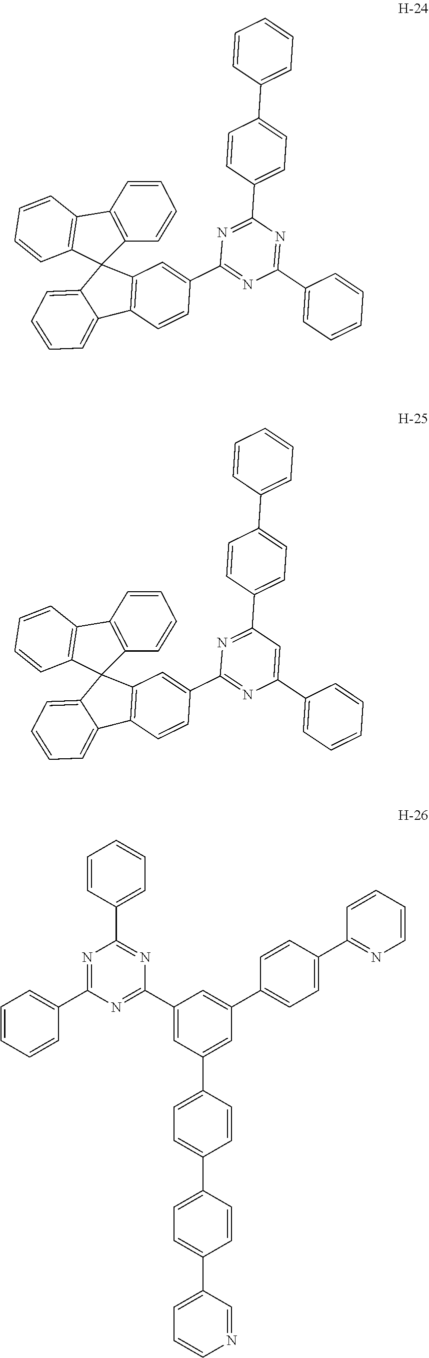 [SMILES: c1ccc(-c2ccc(-c3nc(-c4ccccc4)nc(-c4ccc5c(c4)C4(c6ccccc6-c6ccccc64)c4ccccc4-5)n3)cc2)cc1 ; c1ccc(-c2ccc(-c3cc(-c4ccccc4)nc(-c4ccc5c(c4)C4(c6ccccc6-c6ccccc64)c4ccccc4-5)n3)cc2)cc1 ; c1ccc(-c2nc(-c3ccccc3)nc(-c3cc(-c4ccc(-c5ccc(-c6cccnc6)cc5)cc4)cc(-c4ccc(-c5ccccn5)cc4)c3)n2)cc1]

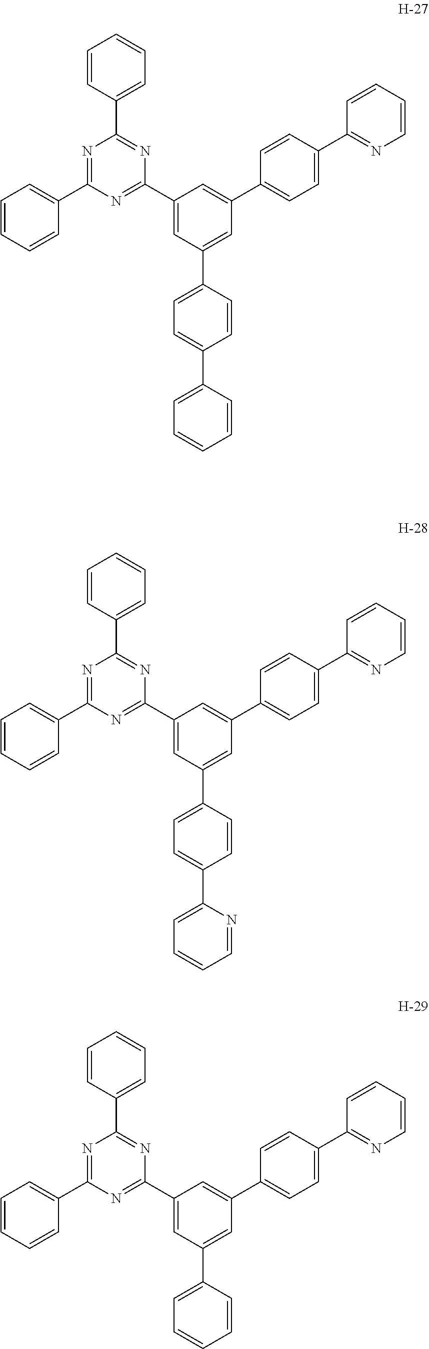 [SMILES: c1ccc(-c2ccc(-c3cc(-c4ccc(-c5ccccn5)cc4)cc(-c4nc(-c5ccccc5)nc(-c5ccccc5)n4)c3)cc2)cc1 ; c1ccc(-c2nc(-c3ccccc3)nc(-c3cc(-c4ccc(-c5ccccn5)cc4)cc(-c4ccc(-c5ccccn5)cc4)c3)n2)cc1 ; c1ccc(-c2cc(-c3ccc(-c4ccccn4)cc3)cc(-c3nc(-c4ccccc4)nc(-c4ccccc4)n3)c2)cc1]

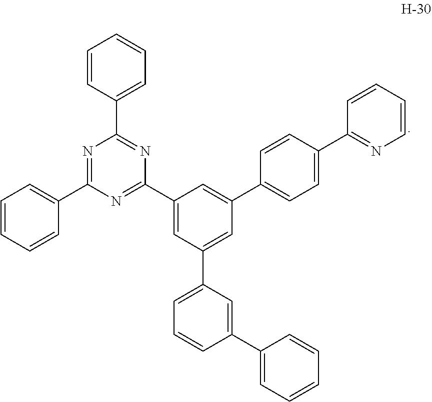 [SMILES: c1ccc(-c2cccc(-c3cc(-c4ccc(-c5ccccn5)cc4)cc(-c4nc(-c5ccccc5)nc(-c5ccccc5)n4)c3)c2)cc1]

In the above organic electroluminescent diode provided by the disclosure, the host material of the light emitting layer is a hole-biased type material, in which the hole migration rate is greater than the electron migration rate. This can cause the electron-hole recombination position to shift towards the side near the cathode. The host material is selected from the compound represented by Chemical Formula 1, so it has relatively high triplet (T1) energy. Based on this, the material of the hole blocking layer of the organic electroluminescent diode in the embodiment of the disclosure is selected from the compound represented by Chemical Formula 2, so that the material of the hole blocking layer also has a relatively high triplet energy, thereby blocking excitons, avoiding or reducing exciton leakage from the light emitting layer to the hole blocking layer, ensuring exciton utilization, and thus ensuring high luminescence efficiency. Furthermore, electrons and holes mainly recombinate on TADF materials, allowing the recombinated excitons to quickly transfer to fluorescent dopant residues for luminescence, avoiding exciton accumulation and material aging.

Moreover, by selecting the host material from the compound represented by Chemical Formula 1 and the material of the hole blocking layer from the compound represented by Chemical Formula 2, the material of the hole blocking layer can have a relatively deep HOMO energy level, effectively blocking holes, reducing the amount of holes transported from the light emitting layer to the hole blocking layer, and constraining the electron-hole recombination position in the light emitting layer. Furthermore, it reduces exciton loss and ensures high luminescence efficiency. Furthermore, the hole blocking layer also protects the film material between the hole blocking layer and the cathode by blocking holes, avoiding accelerated aging of these film materials under the impact of holes.

Moreover, by selecting the host material from the compound represented by Chemical Formula 1 and the material of the hole blocking layer from the compound represented by Chemical Formula 2, the difference in LUMO energy level between the host material and the material of the hole blocking layer can be relatively small, thereby facilitating the injection of electrons from the hole blocking layer into the light emitting layer. This can avoid the accumulation of electrons in the electron transport layer and hole blocking layer, leading to accelerated aging of the material.

It can be seen from the above that in the embodiment of the disclosure, by selecting the host material from the compound represented by Chemical Formula 1 and the material of the hole blocking layer from the compound represented by Chemical Formula 2, the electron-hole recombination position can be shifted to the side of the hole blocking layer, the material of the hole blocking layer has a relatively high triplet energy level to block exciton loss, and the hole blocking layer has a relatively deep HOMO energy level to block holes, the LUMO energy level between the host material and the material of the hole blocking layer is relatively small to facilitate electron injection, and the electron transport layer has a relatively fast electron migration rate and matches the HOMO energy level of the hole blocking layer. Through these characteristics, the electron-hole recombination in the light emitting layer can be efficiently achieved, and the diffusion loss of holes and excitons can be reduced, ensuring the luminescence efficiency. While ensuring high luminescence efficiency, the organic electroluminescent diode according to the embodiment of the disclosure can also reduce the aging rate of the material by avoiding the diffusion of excitons and holes towards the hole blocking layer and the electron transport layer, as well as by avoiding the accumulation of electrons in the hole blocking layer and the electron transport layer, thereby improving the lifetime of the organic electroluminescent diode and effectively reducing the driving voltage, and to some extent, improving the exciton recombination region within the light emitting layer.

In addition, in the organic electroluminescent diode of the embodiment of the disclosure, the light emitting layer further includes TADF material and fluorescent dopant material. Because the host material has a relatively high triplet energy level, the exciton reflow on the TADF material can be reduced or avoided. The TADF material can quickly transform the triplet excitons to the singlet excitons and transfer the singlet excitons to the fluorescent dopant material, so that the fluorescent dopant material can quickly release energy through fluorescence. In this way, in the OLED in the embodiment of the disclosure, energy can be quickly converted, transferred, and released, avoiding material aging caused by energy accumulation, which is beneficial for improving the lifetime of the organic electroluminescent diode.

In an embodiment, the fluorescent dopant material is a fluorescent material containing boron element.

In an embodiment of the disclosure, the content of the host material in the light emitting layer (the proportion of the evaporation rate component of the host material in the coevaporation rate) is not less than 50%.

In an embodiment of the disclosure, the content of fluorescent dopant material in the light emitting layer (the proportion of the evaporation rate component of the fluorescent dopant material in the coevaporation rate) is not greater than 5% to avoid fluorescence quenching. Furthermore, in the light emitting layer, the content of fluorescent dopant materials (the proportion of the evaporation rate component of fluorescent dopant materials in the co evaporation rate) is greater than 0.5%.

In an embodiment of the disclosure, the rate of exciton transfer from the TADF material to the fluorescent dopant material is greater than the quenching rate of the triplet exciton of the TADF material. In this way, on the one hand, it can improve the purity of the light emitted, and on the other hand, it can reduce the aging of various film materials in the light emitting layer, thereby improving the lifetime of the organic electroluminescent diode.

In one example, the energy level difference between the first singlet energy level and the first triplet energy level of the TADF material is not more than 0.2 eV, so as to ensure that the TADF material can effectively use the exciton of the first triplet energy level.

In an embodiment of the disclosure, the TADF material content (the proportion of the evaporation rate component of the fluorescent dopant material in the coevaporation rate) in the light emitting layer is greater than 5% and less than 50%.

In an embodiment of the disclosure, the luminescence efficiency of TADF material in the light emitting layer accounts for less than 10% of the total luminescence efficiency of the electromechanical electroluminescent diode. The energy of TADF materials is mainly transferred to fluorescent dopant materials to ensure their luminescence.

In an embodiment of the disclosure, in the light emitting layer, the first triplet energy level of the fluorescent dopant is lower than the first triplet energy level of the TADF material, and the first singlet energy level of the fluorescent dopant is lower than the first singlet energy level of the TADF material, so as to ensure that excitons in the TADF material can be transferred to the fluorescent dopant material, and avoid exciton reflow.

In an embodiment of the disclosure, the HOMO (highest occupied molecular orbital) energy level of the host material is greater than −6.55 eV and less than −5.75 eV. The LUMO energy level of the host material is greater than −3.2 eV and less than −2.4 eV.

In an embodiment of the disclosure, the absolute value of the energy level difference between the HOMO level of the material of the hole blocking layer and the HOMO level of the host material is not less than 0.15 eV. The absolute value of the energy level difference between the first triplet energy level of the hole blocking material and the first triplet energy level of the host material is not greater than 0.15 eV.

In one example, the HOMO energy level of the material of the hole blocking layer is smaller than that of the host material.

In one example, the first triplet energy level of the material of the hole blocking layer is slightly lower than the first triplet energy level of the host material, which can not only make the hole blocking layer play a certain role in exciton blocking, but also avoid that the first triplet energy level of the material of the hole blocking layer is too high, resulting in too low electron migration rate, reaching a balance between exciton blocking and improving electron injection efficiency.

In an embodiment of the disclosure, the first triplet energy level of the material of the hole blocking layer is less than the first triplet energy level of the host material, and the absolute value of the energy level difference between the first triplet energy level of the material of the hole blocking layer and the first triplet energy level of the host material is less than 0.1 eV.

In an embodiment of the disclosure, the electron migration rate of the material of the hole blocking layer is not less than 5×10⁻⁶ cm²/Vs. This can ensure that the hole blocking layer has a certain amount of electron transfer ability, avoiding the weak electron transfer ability of the hole blocking layer, which leads to low injection efficiency of electrons into the light emitting layer and accumulation of electrons in the electron transfer layer, thereby ensuring the luminescence efficiency and lifetime of the organic electroluminescent diode.

In an embodiment of the disclosure, the absolute value of the energy level difference between the LUMO (lowest unoccupied molecular orbit) energy level of the material of the hole blocking layer and the LUMO energy level of the host material is less than 0.4 eV. This can ensure that the hole blocking layer can inject electrons into the light emitting layer normally, avoiding the significant difference between the LUMO energy levels of the material of the hole blocking layer and the host material. For example, it can prevent the LUMO energy levels of the material of the hole blocking layer from being too deep, resulting in low efficiency for the hole blocking layer to inject electrons into the light emitting layer and increased driving voltage.

In one embodiment of the disclosure, the thickness of the hole blocking layer is not greater than 10 nm, especially not greater than 5 nm. Optionally, the thickness of the hole blocking layer is between 2 nm to 10 nm, such as 2 nm to 5 nm. This can ensure the normal injection of electrons and improve electron transport performance.

In an embodiment of the disclosure, the absolute value of the energy level difference between the LUMO energy level of the material of the electron transport layer and the LUMO energy level of the material of the hole blocking layer is less than 0.5 eV. The first triplet energy level of the material of the electron transport layer is smaller than the first triplet energy level of the material of the hole blocking layer. This can enable electrons from the electron transport layer to be smoothly injected into the hole blocking layer, thereby reducing the driving voltage and improving the luminescence efficiency.

In an embodiment of the disclosure, the difference between the electron migration of the electronic transport layer and the electron migration rate of the hole blocking layer is not more than two orders of magnitude, for example, the electron migration rate of the electronic transport layer is not more than 100 times of the electron migration rate of the hole blocking layer. In particular, the electron migration rate of the electronic transport layer is within the same order of magnitude as the electron migration rate of the hole blocking layer, for example, the electron migration rate of the electronic transport layer does not exceed 10 times the electron migration rate of the hole blocking layer. This allows the electrons in the electron transport layer to be injected into the hole blocking layer smoothly, so as to avoid the problem of electron accumulation caused by the large difference in electron migration rate between the electron transport layer and the hole blocking layer, and thus avoid the problem of efficiency degradation and material aging caused by electron accumulation.

In an embodiment of the disclosure, as shown in FIG. 1, the electron transfer layer includes a first electron transfer layer ETL1 and a second electron transfer layer ETL2 disposed in layers. The first electron transfer layer ETL1 is located on the side of the hole blocking layer away from the second electron transfer layer ETL2. The LUMO energy levels of the hole blocking layer, the second electron transport layer ETL2, and the ELT1 sequentially decrease. This can further reduce the driving voltage and improve the luminescence efficiency.

In an embodiment of the disclosure, the electron transfer layer includes a first electron transfer layer ETL1 and a second electron transfer layer ETL2 disposed in layers, and the first electron transfer layer ETL1 is located on the side of the hole blocking layer away from the second electron transfer layer ETL2. The first triplet energy levels of the hole blocking layer, the second electron transport layer ETL2 and the ELT1 sequentially decrease. In some cases, this can sequentially increase the electron migration rate of the hole blocking layer, the second electron transport layer ETL2 and the ELT1, thereby further reducing the driving voltage and improving the luminescence efficiency.

In one embodiment of the disclosure, the thickness of the light emitting layer ranges from 10 nm to 30 nm.

In one embodiment of the disclosure, the thickness of the electron transport layer is between 20 nm and 70 nm.

In one embodiment of the disclosure, as shown in FIG. 1, the organic electroluminescent diode further comprises a hole transport layer HTL, which is located between the anode and the light emitting layer to transport holes. The material of the hole transport layer can have a high hole migration rate.

In one example, the HOMO energy level of the material of the hole transport layer is between −5.2 eV and −5.6 eV.

In one example, the material of the hole transport layer can be materials with high hole migration such as triarylamine materials, carbazole materials, etc.

In one example, the thickness of the hole transport layer can range from 100 nm to 140 nm.

In an embodiment of the disclosure, as shown in FIG. 1, the organic electroluminescent diode may further include a hole injection layer HIL located between the hole transport layer and the anode. The hole injection layer is used to reduce the hole injection potential blocking and improve the efficiency of the anode injecting holes into the hole transport layer.

In one example, the material of the hole injection layer can be selected from triarylamine-based material, carbazole-based material, and the like.

In another example, the material of the hole injection layer can be selected from the P-type doped hole transport layer material, such as NPB: F4TCNQ, TAPC: MnO3, and the like. Furthermore, the content of the dopant ranges from 0.5% to 10%.

In one example, the thickness of the hole injection layer is 5 nm to 20 nm.

In an embodiment of the disclosure, as shown in FIG. 1, the organic electroluminescent diode may further include an electron blocking layer EBL, which is used to inject holes into the light emitting layer and block the diffusion of electrons and excitons from the light emitting layer towards the hole transport layer.

In one example, the thickness of the electron blocking layer is 1 nm to 10 nm.

In an embodiment of the disclosure, as shown in FIG. 1, the organic electroluminescent diode may further include an electron injection layer EIL, which is used to improve the efficiency of electron injection from the cathode to the electron transport layer.

In one example, the thickness of the electron injection layer can be between 0.5 nm to 2 nm.

In one embodiment of the disclosure, the anode may use a material with a high work function.

In one example, the organic electroluminescent diode has a bottom emitting structure, where the light emitted from the emitting layer is emitted through the anode. The anode can use transparent metal oxides, such as ITO (indium tin oxide), IZO (indium zinc oxide), and other materials. Furthermore, the thickness of the anode ranges from 80 nm to 200 nm.

In another example, the organic electroluminescent diode have a top emitting structure, where the light emitted from the emitting layer is emitted through the cathode, and the anode can be a composite structure of a reflective layer/transparent metal oxide layer. Wherein, the reflective layer is located on the side of the transparent metal oxide layer away from the light emitting layer. For example, the anode can be a composite structure of Ag (as a reflective layer)/ITO or Ag (as a reflective layer)/IZO. Furthermore, the thickness of the reflective layer is between 80 nm and 100 nm. The thickness of the transparent metal oxide layer is 5 nm to 10 nm, and the average reflectivity of the anode in the visible light region is 85%˜95%.

In one embodiment of the disclosure, the material or thickness of the cathode can be determined as needed.

When the organic electroluminescent diode have a top emitting structure, the light emitted by the emitting layer needs to be emitted from the cathode, and at this time, the cathode can use a transparent metal electrode. In one example, the cathode can be a 10 nm to 20 nm magnesium layer, silver layer, aluminum layer, and the like, or a magnesium silver alloy layer can be used. Wherein, the ratio of magnesium to silver in the magnesium silver alloy layer ranges from 3:7 to 1:9. In one example, the cathode has a transmittance of 50% to 60% at 530 nm.

When the organic electroluminescent diode has a bottom emitting structure, the cathode can use a thicker metal layer to ensure good reflectivity of the cathode. For example, the cathode can include a silver or aluminum layer exceeding 80 nm.

The embodiment of the disclosure also provides a display panel with multiple the organic electroluminescent diodes as described in the aforementioned embodiments.

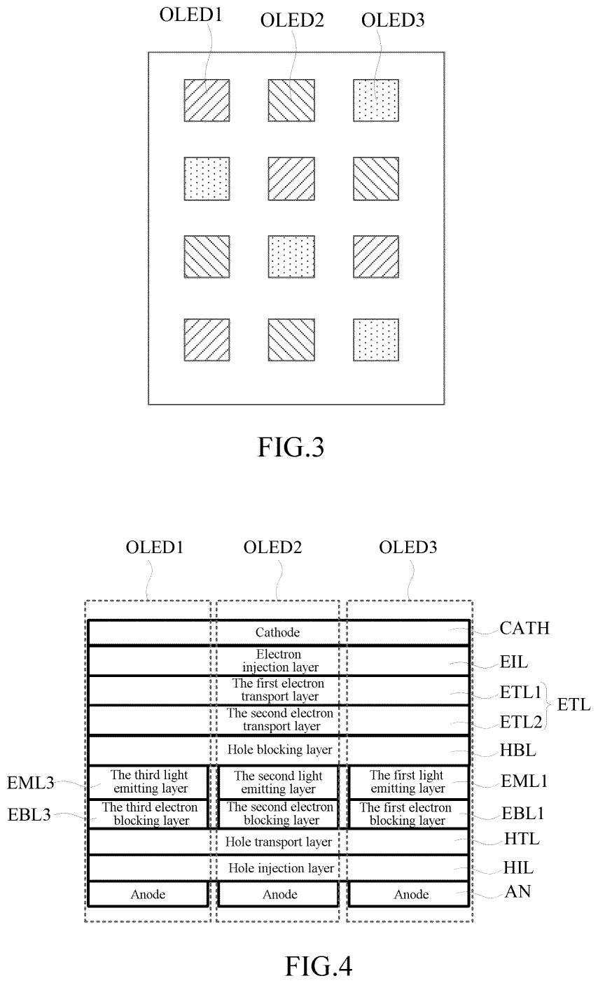

In an embodiment of the disclosure, as shown in FIGS. 2 and 3, the display panel includes a substrate BP, a driving circuit layer F100, a pixel layer F200, and a packaging layer TFE disposed in sequence. The pixel layer F200 is provided with an organic electroluminescent diode as a sub pixel (such as the first organic electroluminescent diode OLED1, the second organic electroluminescent diode OLED2, and the third organic electroluminescent diode OLED3 in FIG. 2). The driving circuit layer is disposed with a pixel driving circuit PDC that drives the organic electroluminescent diode.

In one embodiment of the disclosure, as shown in FIGS. 2 to 4, the pixel layer is disposed with various the organic electroluminescent diodes of different colors (such as the first organic electroluminescent diode OLED1, the second organic electroluminescent diode OLED2, and the third organic electroluminescent diode OLED3), and the light emitting layers of the organic electroluminescent diodes of different colors are different. For example, the emitting layer of the first organic light emitting diode OLED1 is the first emitting layer EML1, the emitting layer of the second organic light emitting diode OLED2 is the second emitting layer, and the emitting layer of the third organic light emitting diode OLED3 is the third emitting layer EML3.

In one example, the hole blocking layer and electron transport layer of each organic electroluminescent diode are the same, in order to reduce the cost of the display panel by preparing these film layers through an open mask.

In one example, the electron injection layer, hole injection layer, and hole transport layer of each organic electroluminescent diode are the same, in order to reduce the cost of the display panel by preparing these film layers through an open mask.

Optionally, the electronic blocking layer of the organic electroluminescent diodes of different colors can be the same or different. As one example, the electron blocking layers of the organic electroluminescent diode of different colors can be matched with the emitting layers of the organic electroluminescent diode to achieve more corresponding and better electron blocking effects. For example, the electron blocking layer of the first organic light emitting diode OLED1 is the first electron blocking layer EBL1, the electron blocking layer of the second organic light emitting diode OLED2 is the second electron blocking layer, and the electron blocking layer of the third organic light emitting diode OLED3 is the third electron blocking layer EBL3.

In one embodiment of the disclosure, the host materials may be the same or different in the light emitting layers of the organic electroluminescent diode of different colors.

In one embodiment of the disclosure, the TADF materials may be the same or different in the light emitting layers of the organic electroluminescent diode of different colors.

In one embodiment of the disclosure, the fluorescent dopant materials in the light emitting layers of the organic electroluminescent diode of different colors may be different.

In one embodiment of the disclosure, each organic electroluminescent diode on the display panel is the organic electroluminescent diode described in the aforementioned embodiments.

In another embodiment of the disclosure, among the various the organic electroluminescent diode on the display panel, only a portion of them use the organic electroluminescent diode described in the aforementioned embodiments, for example, only the red light organic electroluminescent diode uses the organic electroluminescent diode described in the aforementioned embodiments.

As follows, the embodiments of the disclosure disclose the structures and test results of several the organic electroluminescent diodes. These organic electroluminescent diodes include test devices (test devices 1 to 5) prepared using the organic electroluminescent diode design scheme of the disclosure, and control devices (control devices 1 to 4) not prepared according to the organic electroluminescent diode design scheme of the disclosure. The thickness of each film layer of the test devices and the control devices is the same. Except for the light emitting layer, the hole blocking layer, and the electron transport layer, the materials of each film layer are also the same. In this case, the performance differences of each device come from the material matching differences between the light emitting layer, the hole blocking layer, and the electron transport layer.

In these experiments, compounds RA-1 and RA-2 were introduced as the host materials in the light emitting layers of the control devices. Compounds RH-1 and RH-2 were introduced as materials of the hole blocking layers for control devices. In these experiments, the TADF material is compound B1, and the fluorescent dopant material is compound C1. In the electron transport layer, compound E2 is doped to improve the electron migration rate.

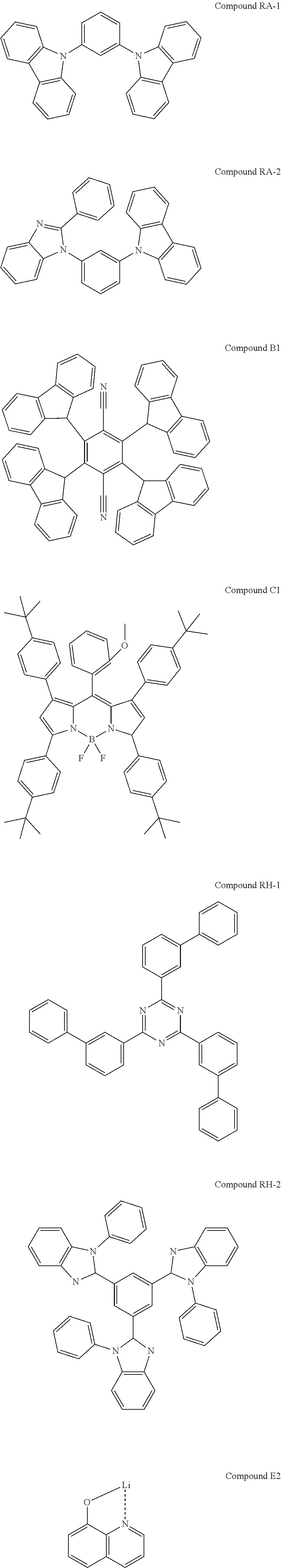

The layered structure of Test Device 1 is:
ITO/HIL/HTL/EBL/EML(A-2:B1:C1)/HBL(H-15)/ETL(H-27: E2)/EIL/Al

The layered structure of Test Device 2 is:
ITO/HIL/HTL/EBL/EML(A-6:B1:C1)/HBL(H-15)/ETL(H-27:E2)/EIL/Al

The layered structure of Test Device 3 is:
ITO/HIL/HTL/EBL/EML(A-11:B1:C1)/HBL(H-15)/ETL(H-27: E2)/EIL/Al

The layered structure of Test Device 4 is:
ITO/HIL/HTL/EBL/EML (A-2:B1:C1)/HBL (H-15)/ETL(H-9: E2)/EIL/Al

The layered structure of Test Device 5 is:
ITO/HIL/HTL/EBL/EML(A-2:B1:C1)/HBL (H-15)/ETL(H-5)/ETL(H-27: E2)/EIL/Al

The layered structure of Control Device 1 is:
ITO/HIL/HTL/EBL/EML (RA-1: B1: C1)/HBL (RH-1)/ETL (RH-2: E2)/EIL/Al

The stacked structure of Control Device 2 is:
ITO/HIL/HTL/EBL/EML (RA-2:B1:C1)/HBL(RH-1)/ETL (RH-2:E2)/EIL/Al

The layered structure of Control Device 3 is:
ITO/HIL/HTL/EBL/EML(RA-1:B1:C1)/HBL(RH-2)/ETL(RH-2:E2)/EIL/Al

The layered structure of Control Device 4 is:
ITO/HIL/HTL/EBL/EML(RA-1:B1:C1)/HBL(H-15)/ETL(H-27:E2)/EIL/A1

The devices of the above examples are a bottom light emitting device. Wherein, the thickness of the hole injection layer HIL is 10 nm, the hole transfer layer HTL is 100 nm, the electron blocking layer EBL is 5 nm, the light emitting layer EML is 25 nm, the hole blocking layer HBL is 5 nm, the electron transfer layer ETL is 35 nm, the electron injection layer EIL is 1 nm, and the cathode (Al) is 120 nm.

The physical property data of each compound involved are as follows:

TABLE 1
Physical Property Data of Host Materials

| | | | | Hole | Electron |
|---|---|---|---|---|---|
| | HOMO | LUMO | T1 | migration rate | migration rate |
| Compound | (eV) | (eV) | (eV) | (cm²/V S) | (cm²/V S) |
| A-2 | −6.11 | −2.61 | 2.85 | 1.15 × 10⁻⁷ | — |
| A-6 | −5.92 | −2.52 | 2.90 | 1.94 × 10⁻⁷ | — |
| A-11 | −6.10 | −2.65 | 2.82 | 1.62 × 10⁻⁷ | — |
| RA-1 | −5.90 | −2.43 | 3.0 | 1.32 × 10⁻⁶ | — |
| RA-2 | −5.82 | −2.25 | 3.0 | 4.32 × 10⁻⁵ | 6.12 × 10⁻⁵ |

TABLE 2
Physical Property Data of Materials of Hole
Blocking Layer and Electron Transport Layer

| | HOMO | LUMO | T1 | Electron migration rae |
|---|---|---|---|---|
| Compound | (eV) | (eV) | (eV) | (cm²/V S) |
| H-5 | −6.57 | −3.0 | 2.65 | 1.5 × 10⁻⁴ |
| H-9 | −6.30 | −3.0 | 2.6 | 1.2 × 10⁻⁵ |
| H-15 | −6.28 | −2.8 | 2.8 | 6.6 × 10⁻⁵ |
| H-27 | −6.65 | −3.2 | 2.55 | 3.7 × 10⁻⁵ |
| RH-1 | −6.50 | −3.0 | 2.8 | 1.2 × 10⁻⁴ |
| RH-2 | −6.20 | −2.7 | 2.55 | 0.8 × 10⁻⁵ |

TABLE 3
Test Data for Each Device (Normalized Data)

| | Current | | | | |
|---|---|---|---|---|---|
| | density | Volt- | Effi- | Color | |
| Device | (mA/cm²) | age | ciency | Coordinate | LT95 |
| Test Device 1 | 15 | 100% | 100% | (0.64, 0.36) | 100% |
| Test Device 2 | | 98% | 109% | (0.64, 0.36) | 124% |
| Test Device 3 | | 98% | 128% | (0.64, 0.36) | 119% |
| Test Device 4 | | 102% | 99% | (0.64, 0.36) | 99% |
| Test Device 5 | | 97% | 106% | (0.64, 0.36) | 110% |
| Control Device 1 | | 102% | 102% | (0.64, 0.36) | 67% |
| Control Device 2 | | 97% | 101% | (0.64, 0.36) | 78% |
| Control Device 3 | | 105% | 85% | (0.64, 0.36) | 58% |
| Control Device 4 | | 103% | 98% | (0.64, 0.36) | 99% |

According to Table 3, it can be found that the lifetime of control devices 1 to 3 is significantly lower than that of test devices 1 to 5. This indicates that in the case of using the same structure but different materials, the organic electroluminescent diodes of the disclosure can significantly reduce material aging and improve device lifetime. The main reason for this is that the disclosure uses specific host materials, materials of the hole blocking layer, and materials of electron transport layer to match the energy levels and carrier migration rates between each material, thereby reducing the aging rate of the material.

In the above-mentioned control device, the host material RA-1 used has a symmetrical structure and two carbazole groups, which makes the electron tolerance of compound RA-1 poor and easily leads to a decrease in device lifetime due to aging. Therefore, in the control devices 1 and 3, if the design concept of the electron blocking layer and electron transport layer is not improved according to the embodiment of the disclosure, the material disadvantage of compound RA-1 will be clearly presented, resulting in poor device lifetime of the control devices 1 and 3. In the control device 4, the design concept of the electronic blocking layer and the electronic transport layer in the embodiment of the disclosure is adopted to improve the device, resulting in a significant increase in the device lifetime of the control device 4. In other words, although compound RA-1 itself is prone to a decrease in device lifetime due to aging, the design concept of the electron blocking layer and the electron transport layer in the embodiment of the disclosure can compensate for the shortcomings of compound RA-1. This further indicates that the matching selection between the host material, the electron blocking layer, and the electron transport layer of the organic electroluminescent diode in the embodiment of the disclosure has achieved unexpected effects, which can significantly improve the lifetime of the device. Of course, the inventors found that RA-1 has other defects when applied to the light emitting layer, such as poor film-forming property and low glass transition temperature due to good molecular symmetry and small molecular weight, which will reduce the preparability of organic electroluminescent diodes.

In the above-mentioned devices, compound RA-2 is a bipolar material, which can cause electron-hole recombination on RA-2, leading to its easy aging.

In the above-mentioned devices, the migration mismatch between the hole blocking material RH-1 and the electron transfer material RH-2 causes electrons to accumulate at the interface between the light emitting layer and the hole blocking layer, accelerating the aging of the interface material and reducing the device lifetime. If RH-2 is used as the material of the hole blocking, its first triplet energy level is low, which is easy to lead to exciton leakage, resulting in reduced exciton utilization, leading to a significant decline in the efficiency of electroluminescent devices.

Those skilled in the art will easily think of other embodiments of the disclosure after considering the specification and practicing the content disclosed herein. The present application is intended to cover any variations, uses, or adaptive changes of the present disclosure. These variations, uses, or adaptive changes follow the general principle of the present disclosure and include common knowledge or conventional technical means in the technical field that are not disclosed in the present disclosure. The description and the embodiments are only regarded as exemplary, and the true scope and spirit of the present disclosure are limited by the appended claims.

1. An organic electroluminescent diode, comprising an anode, a light emitting layer, a hole blocking layer, an electron transport layer, and a cathode, which are stacked in sequence: wherein the light emitting layer comprises a host material, a TADF material, and a fluorescent dopant material:
the host material is selected from the compound represented by Chemical Formula 1, and the material of the hole blocking layer is selected from the compound represented by Chemical Formula 2: 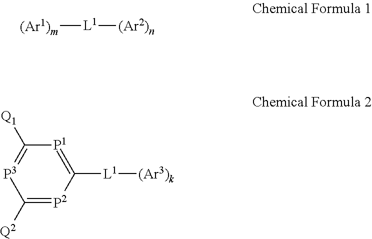
wherein, m and n are the same or different, and are independently selected from an integer not less than 1; K is 1 or 2;
L¹ is selected from a single bond, and substituted or unsubstituted aryl group with 6 to 12 ring-forming carbon atoms. In a case that L¹ has a substituent, the substituent is selected from deuterium, fluorine, cyano, alkyl group with 1 to 4 carbon atoms, deuteroalkyl group with 1 to 4 carbon atoms, fluoroalkyl group with 1 to 4 carbon atoms, and aryl group with 6 to 12 ring-forming carbon atoms:
Ar¹ is selected from the groups shown in Chemical Formulas 1-A, 1-B, and 1-C: 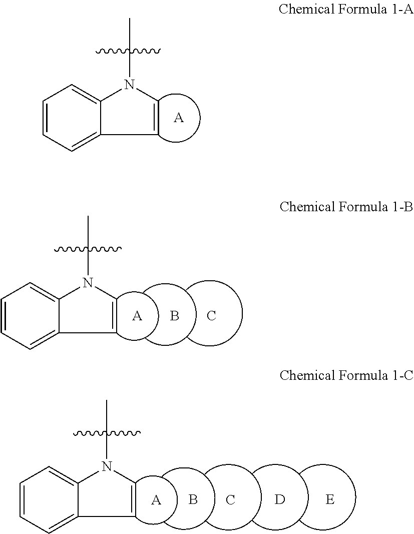
Ring A, Ring C, and Ring E are independently substituted or unsubstituted benzene ring. In a case that Ring A, Ring C, or Ring E has a substituent, the substituent is selected from deuterium, fluorine, cyano, alkyl group with 1 to 4 carbon atoms, deuteroalkyl group with 1 to 4 carbon atoms, and fluoroalkyl group with 1 to 4 carbon atoms: Ring B is 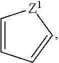
and Ring D is 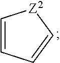
Z¹ and Z² are independently selected from NR1, O, S, C(R²R³), Si(R²R³), and Ge(R²R³); wherein, R¹, R², and R³ are the same or different, and each independently selected from hydrogen, alkyl group with 1 to 4 carbon atoms, and aryl group with 6 to 12 ring-forming carbon atoms:
Ar² is selected from the groups shown in Chemical Formulas 1-D, 1-E, 1-F, and 1-G: 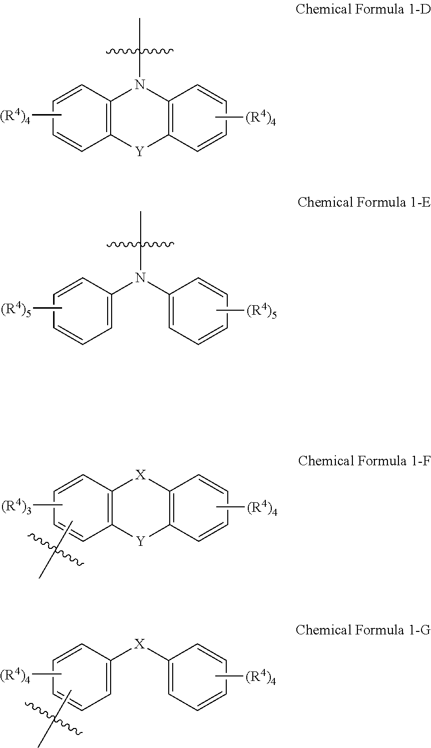
each R⁴ is the same or different, and is independently selected from hydrogen, deuterium, fluorine, cyano, alkyl group with 1 to 4 carbon atoms, deuteroalkyl group with 1 to 4 carbon atoms, fluoroalkyl group with 1 to 4 carbon atoms, aryl group with 6 to 12 ring-forming carbon atoms, and heteroaryl group with 3 to 15 ring-forming carbon atoms:
X and Y are independently selected from NR⁵, O, S, C(R⁶R⁷), Si(R⁶R⁷), Ge(R⁶R⁷). R⁵, R⁶, and R⁷ are independently selected from hydrogen, alkyl group with 1 to 4 carbon atoms, and aryl group with 6 to 12 ring-forming carbon atoms, and Y can also be a single bond:
and Ar¹ and Ar² are not simultaneously N-carbazolyl group:
L² is selected from a single bond, substituted or unsubstituted aryl group with 6 to 12 ring-forming carbon atoms. In a case that the L² has a substituent, the substituent is selected from deuterium, fluorine, cyano, alkyl group with 1 to 4 carbon atoms, deuteroalkyl group with 1 to 4 carbon atoms, and fluoroalkyl group with 1 to 4 carbon atoms:
P¹, P², and P³ are the same or different, each independently selected from N or CH, and at least two are N:
Ar³ is selected from the groups shown in Chemical Formulas 2-A and 2-B: 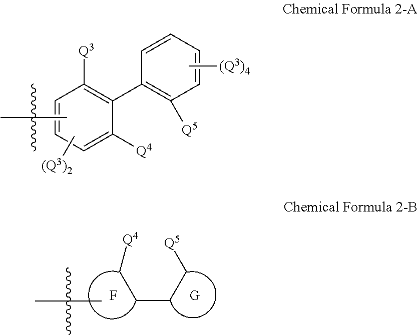
Ring F and Ring G are independently selected from benzene ring or pyridine ring, and at least one is pyridine ring;
Q¹, Q², and each Q³, Q⁴, and Q⁵ are the same or different, and are independently selected from hydrogen, deuterium, cyano, fluorine, substituted or unsubstituted aryl group with 5 to 50 carbon atoms, and substituted or unsubstituted alkyl group with 1 to 50 carbon atoms. Alternatively, Q⁴ and Q⁵ can be condensed to form 5- to 7-membered cycle with connected groups.

2. The organic electroluminescent diode according to claim 1, wherein the material of the electron transport layer is also selected from the compound represented by Chemical Formula 2.

3. The organic electroluminescent diode according to claim 1, wherein the group represented by Chemical Formula 1-B is selected from the following groups: 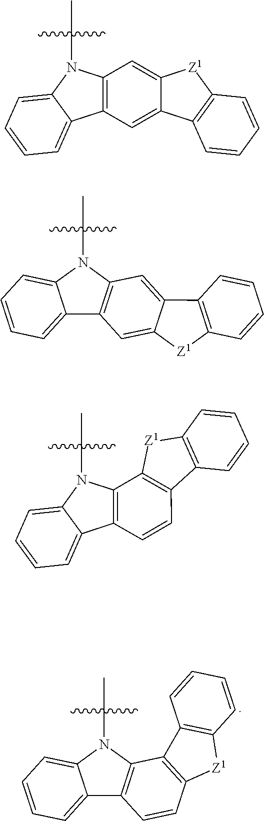
Chemical Formula 1-C is selected from the following groups:

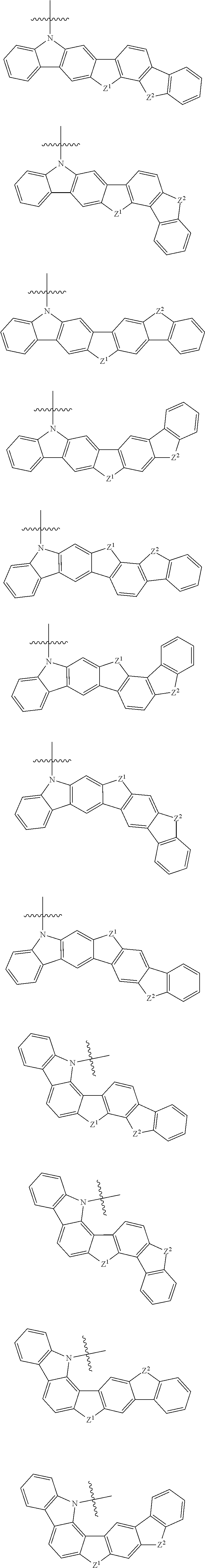

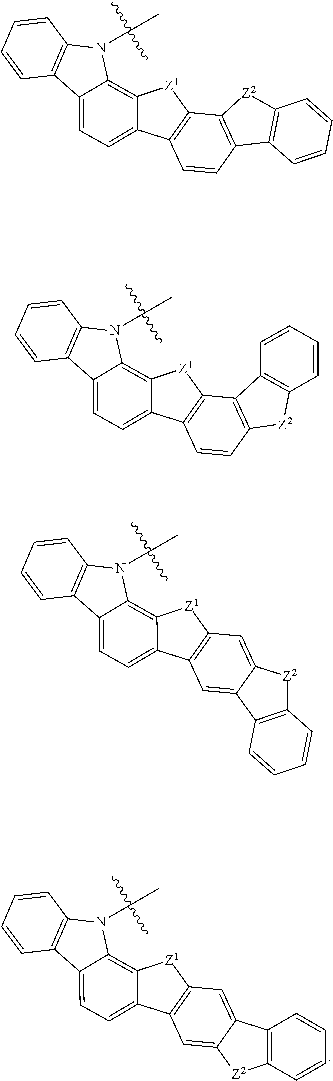

4. The organic electroluminescent diode according to claim 1. wherein Ar¹ is selected from the following groups:

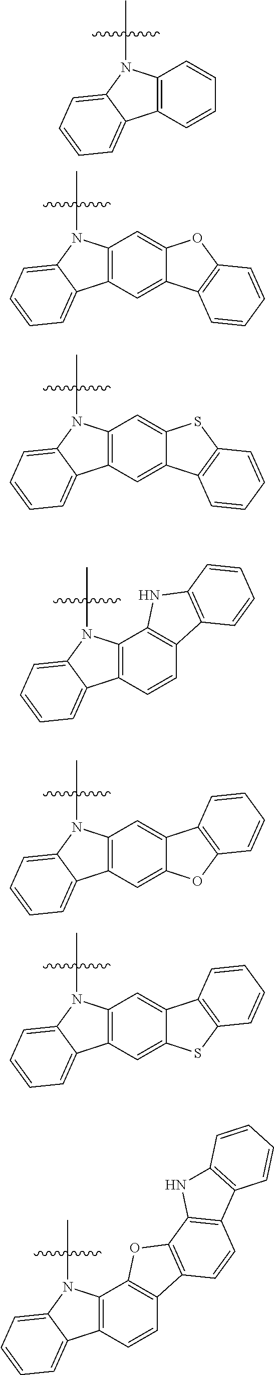

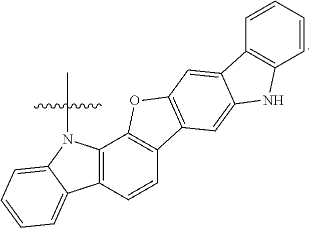 [SMILES: *n1c2ccccc2c2ccc3c4cc5[nH]c6ccccc6c5cc4oc3c21]

5. The organic electroluminescent diode according to claim 1, wherein Ar² is selected from the following groups:

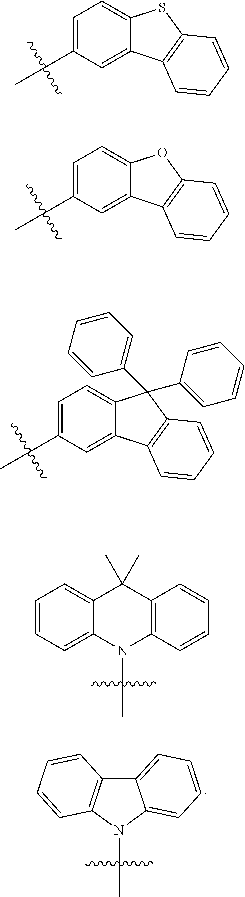 [SMILES: *c1ccc2sc3ccccc3c2c1 ; *c1ccc2oc3ccccc3c2c1 ; *c1ccc2c(c1)-c1ccccc1C2(c1ccccc1)c1ccccc1 ; *N1c2ccccc2C(C)(C)c2ccccc21 ; *n1c2ccccc2c2ccccc21]

6. The organic electroluminescent diode according to claim 1, wherein the compound represented by Chemical Formula 1 is selected from the following groups:

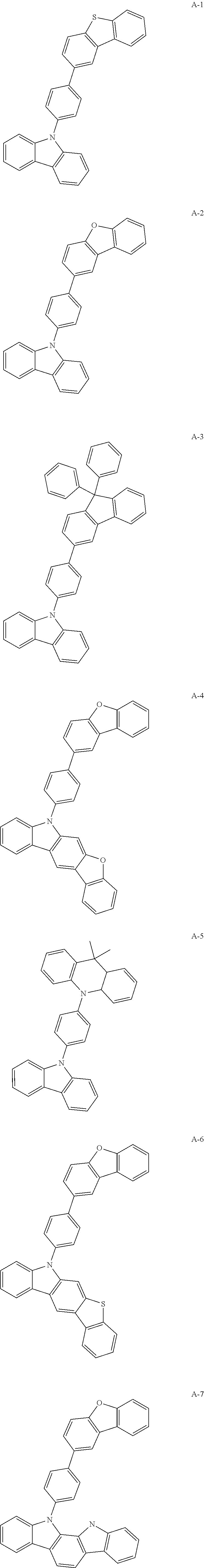

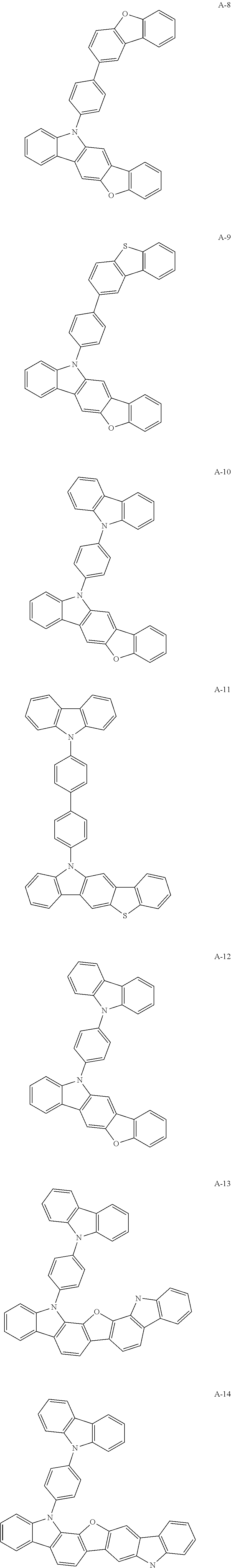

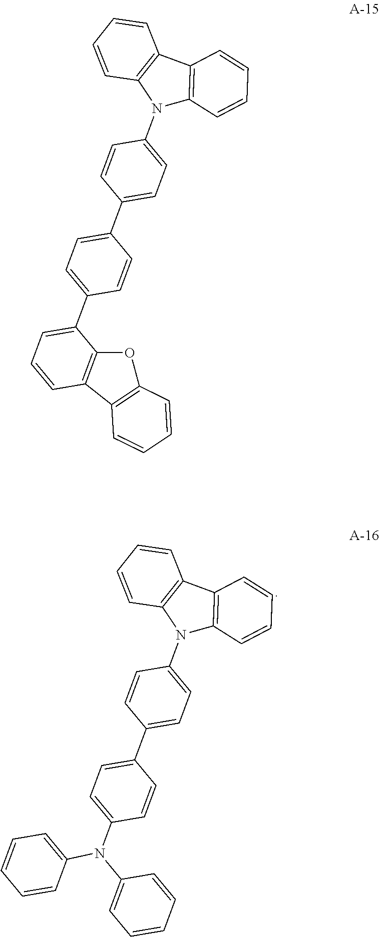 [SMILES: c1ccc2c(c1)oc1c(-c3ccc(-c4ccc(-n5c6ccccc6c6ccccc65)cc4)cc3)cccc12 ; c1ccc(N(c2ccccc2)c2ccc(-c3ccc(-n4c5ccccc5c5ccccc54)cc3)cc2)cc1]

7. The organic electroluminescent diode according to claim 1. wherein Q¹ and Q² are the same or different, and are each independently selected from hydrogen, deuterium, cyano, fluorine, aryl group with 6 to 12 ring-forming carbon atoms.

8. The organic electroluminescent diode according to claim 1, wherein the group represented by Chemical Formula 2-A is selected from the following groups: 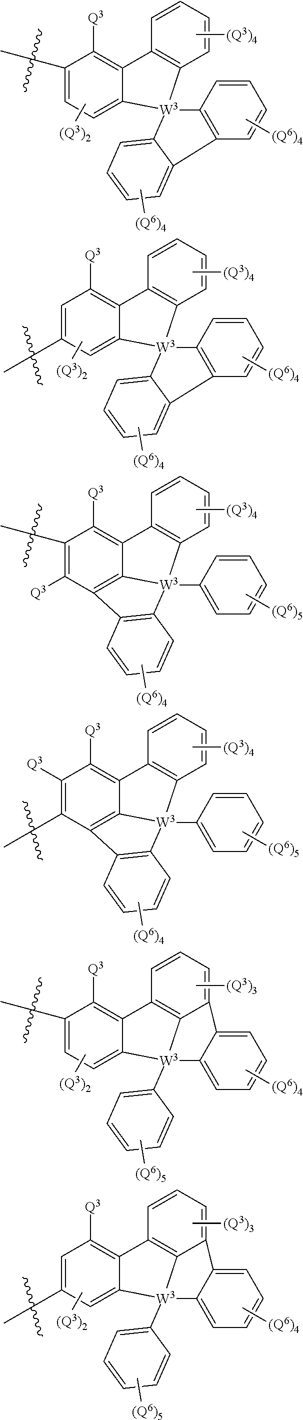 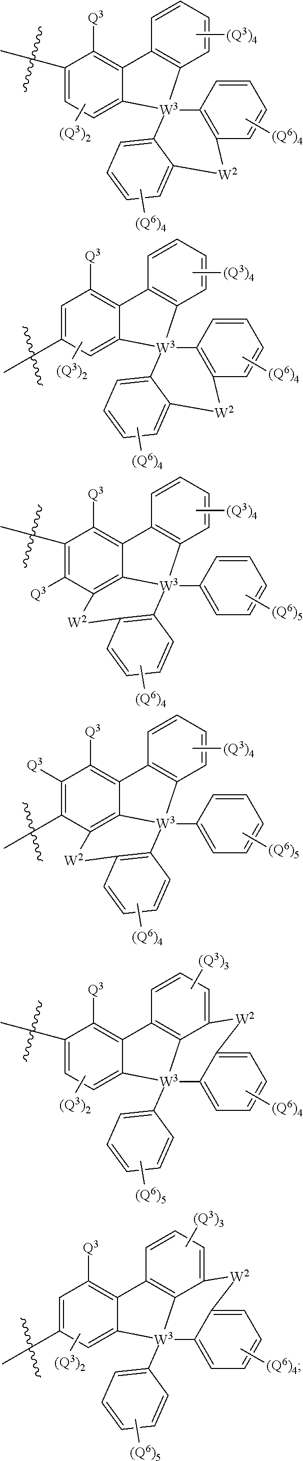
the group represented by Chemical Formula 2-B is selected from the following groups: 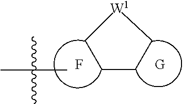
wherein, W¹ is selected from NR⁸, O, and S, and R⁸ is selected from hydrogen, alkyl group with 1 to 4 carbon atoms, and aryl group with 6 to 12 ring-forming carbon atoms;
W² is selected from NR⁹ [symbol] O [symbol] S [symbol] C(R¹⁰R¹¹) [symbol] Si(R¹⁰R¹¹) [symbol] Ge(R¹⁰R¹¹). R⁹ is selected from hydrogen, alkyl group with 1 to 4 carbon atoms, and aryl group with 6 to 12 ring-forming carbon atoms. R¹⁰ and R¹¹ are independently selected from aryl group with 6 to 12 ring-forming carbon atoms;
W³ is selected from C, Si, and Ge;
Each Q⁶ is the same or different, and independently selected from hydrogen, deuterium, cyano, fluorine, substituted or unsubstituted aryl group with 5 to 50 ring-forming carbon atoms, and substituted or unsubstituted alkyl group with 1 to 50 carbon atoms.

9. The organic electroluminescent diode according to claim 1, wherein Ar³ is selected from the following groups:

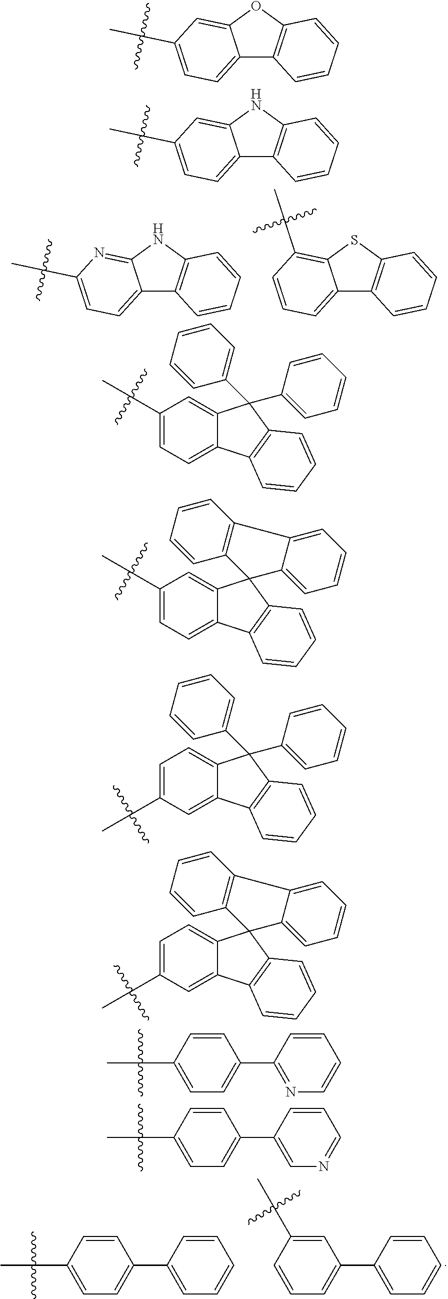

10. The organic electroluminescent diode according to claim 1, wherein the compound represented by Chemical Formula 2 is selected from the following groups:

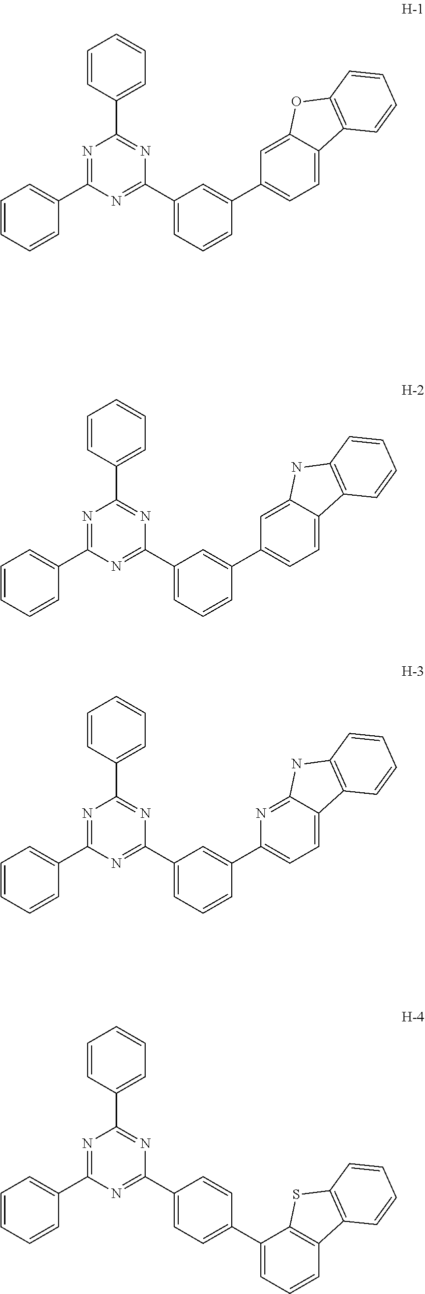 [SMILES: c1ccc(-c2nc(-c3ccccc3)nc(-c3cccc(-c4ccc5c(c4)oc4ccccc45)c3)n2)cc1 ; c1ccc(-c2nc(-c3ccccc3)nc(-c3cccc(-c4ccc5c(c4)[nH]c4ccccc45)c3)n2)cc1 ; c1ccc(-c2nc(-c3ccccc3)nc(-c3cccc(-c4ccc5c(n4)[nH]c4ccccc45)c3)n2)cc1 ; c1ccc(-c2nc(-c3ccccc3)nc(-c3ccc(-c4cccc5c4sc4ccccc45)cc3)n2)cc1]

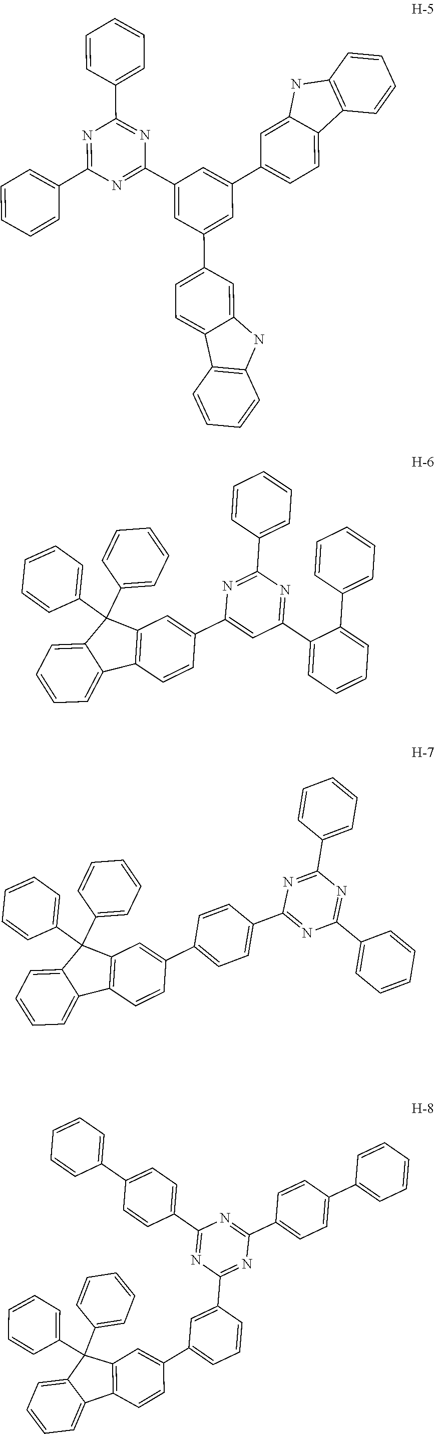

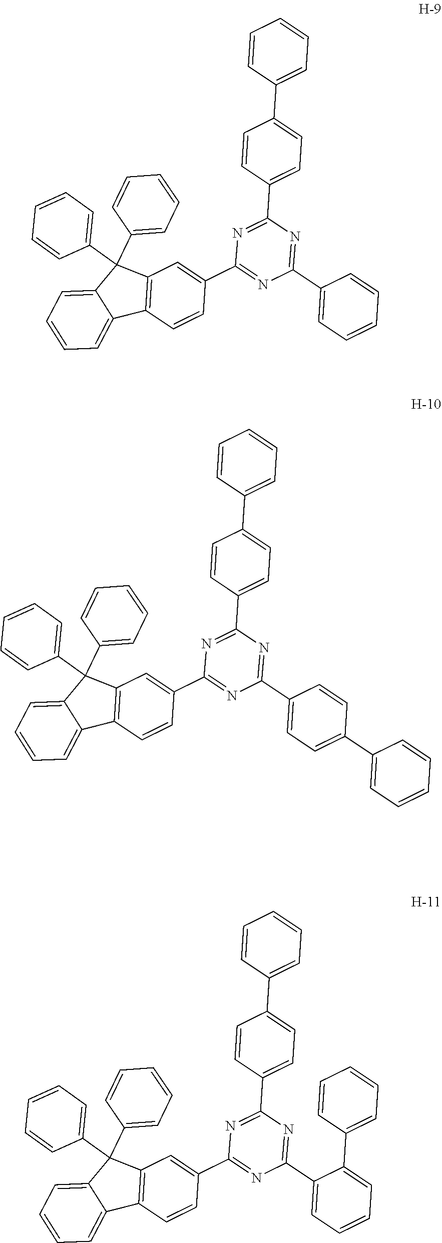 [SMILES: c1ccc(-c2ccc(-c3nc(-c4ccccc4)nc(-c4ccc5c(c4)C(c4ccccc4)(c4ccccc4)c4ccccc4-5)n3)cc2)cc1 ; c1ccc(-c2ccc(-c3nc(-c4ccc(-c5ccccc5)cc4)nc(-c4ccc5c(c4)C(c4ccccc4)(c4ccccc4)c4ccccc4-5)n3)cc2)cc1 ; c1ccc(-c2ccc(-c3nc(-c4ccc5c(c4)C(c4ccccc4)(c4ccccc4)c4ccccc4-5)nc(-c4ccccc4-c4ccccc4)n3)cc2)cc1]

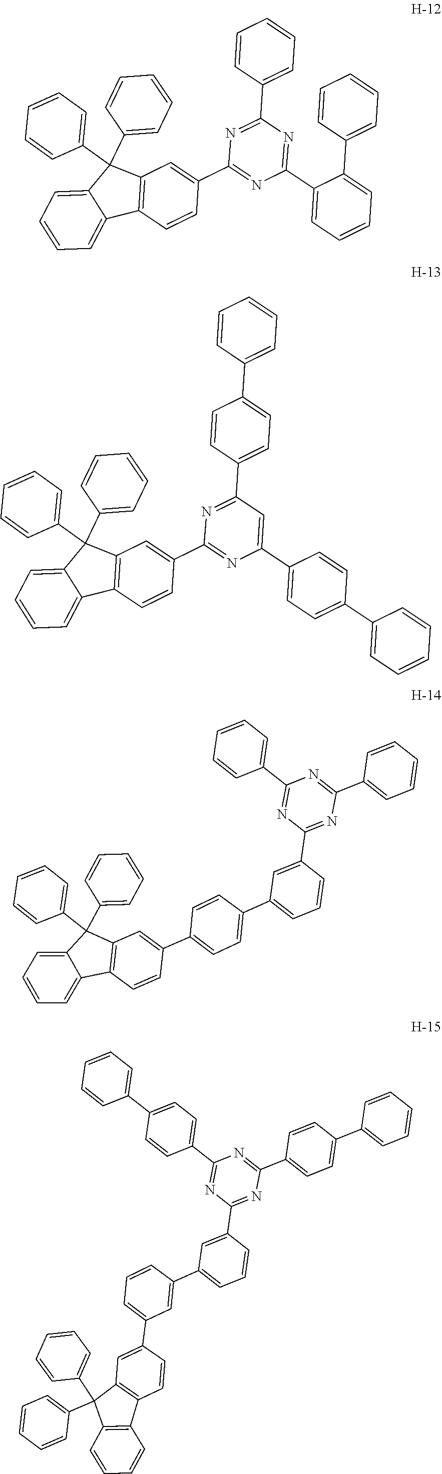 [SMILES: c1ccc(-c2nc(-c3ccc4c(c3)C(c3ccccc3)(c3ccccc3)c3ccccc3-4)nc(-c3ccccc3-c3ccccc3)n2)cc1 ; c1ccc(-c2ccc(-c3cc(-c4ccc(-c5ccccc5)cc4)nc(-c4ccc5c(c4)C(c4ccccc4)(c4ccccc4)c4ccccc4-5)n3)cc2)cc1 ; c1ccc(-c2nc(-c3ccccc3)nc(-c3cccc(-c4ccc(-c5ccc6c(c5)C(c5ccccc5)(c5ccccc5)c5ccccc5-6)cc4)c3)n2)cc1 ; c1ccc(-c2ccc(-c3nc(-c4ccc(-c5ccccc5)cc4)nc(-c4cccc(-c5cccc(-c6ccc7c(c6)C(c6ccccc6)(c6ccccc6)c6ccccc6-7)c5)c4)n3)cc2)cc1]

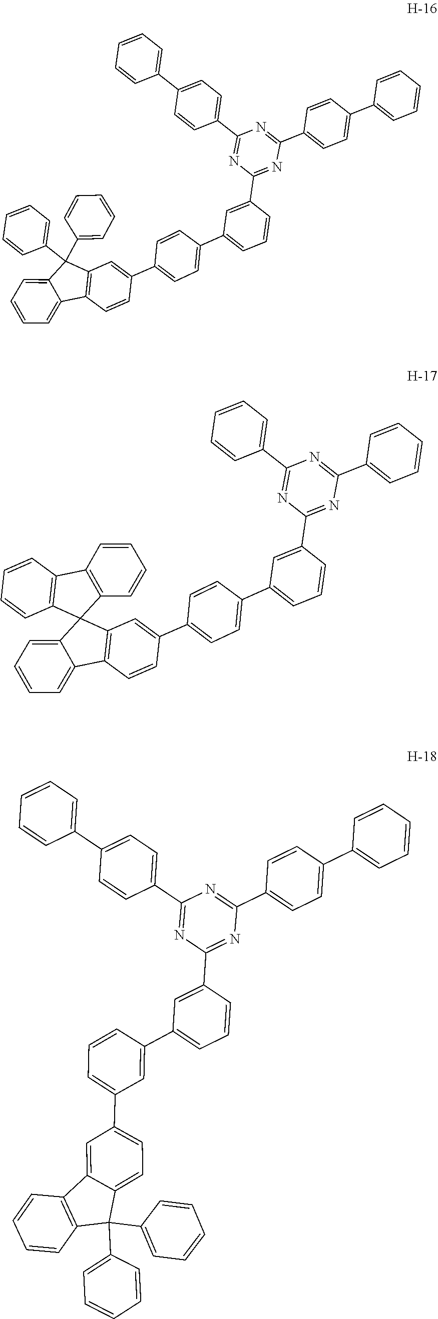 [SMILES: c1ccc(-c2ccc(-c3nc(-c4ccc(-c5ccccc5)cc4)nc(-c4cccc(-c5ccc(-c6ccc7c(c6)C(c6ccccc6)(c6ccccc6)c6ccccc6-7)cc5)c4)n3)cc2)cc1 ; c1ccc(-c2nc(-c3ccccc3)nc(-c3cccc(-c4ccc(-c5ccc6c(c5)C5(c7ccccc7-c7ccccc75)c5ccccc5-6)cc4)c3)n2)cc1 ; c1ccc(-c2ccc(-c3nc(-c4ccc(-c5ccccc5)cc4)nc(-c4cccc(-c5cccc(-c6ccc7c(c6)-c6ccccc6C7(c6ccccc6)c6ccccc6)c5)c4)n3)cc2)cc1]

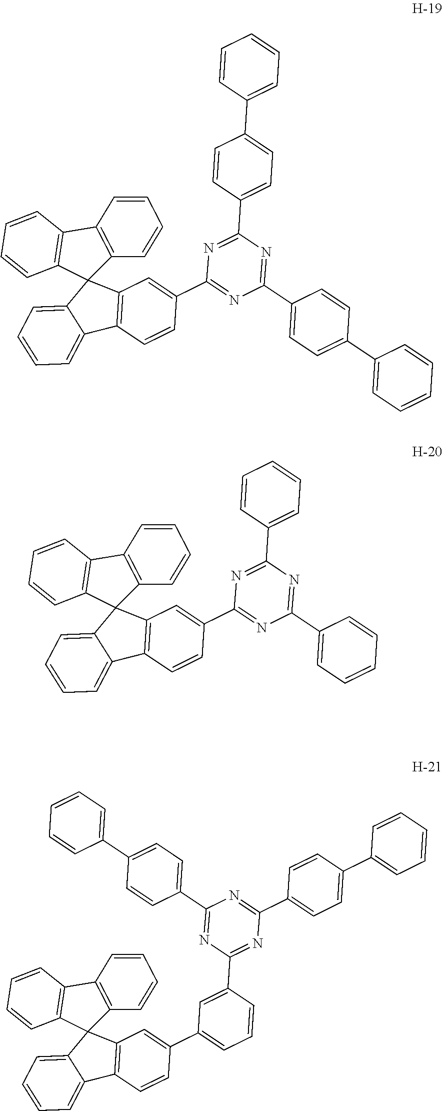 [SMILES: c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1 ; c1ccc(-c2nc(-c3ccccc3)nc(-c3ccc4c(c3)C3(c5ccccc5-c5ccccc53)c3ccccc3-4)n2)cc1 ; c1ccc(-c2ccc(-c3nc(-c4ccc(-c5ccccc5)cc4)nc(-c4cccc(-c5ccc6c(c5)C5(c7ccccc7-c7ccccc75)c5ccccc5-6)c4)n3)cc2)cc1]

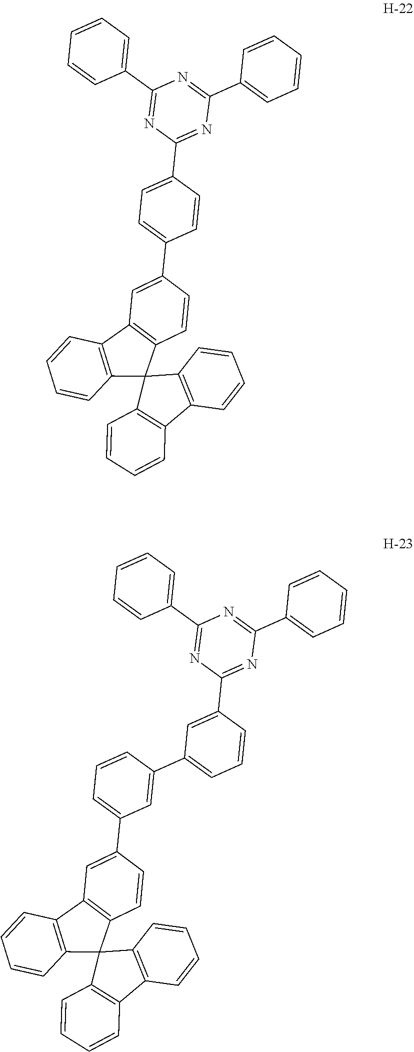 [SMILES: c1ccc(-c2nc(-c3ccccc3)nc(-c3ccc(-c4ccc5c(c4)-c4ccccc4C54c5ccccc5-c5ccccc54)cc3)n2)cc1 ; c1ccc(-c2nc(-c3ccccc3)nc(-c3cccc(-c4cccc(-c5ccc6c(c5)-c5ccccc5C65c6ccccc6-c6ccccc65)c4)c3)n2)cc1]

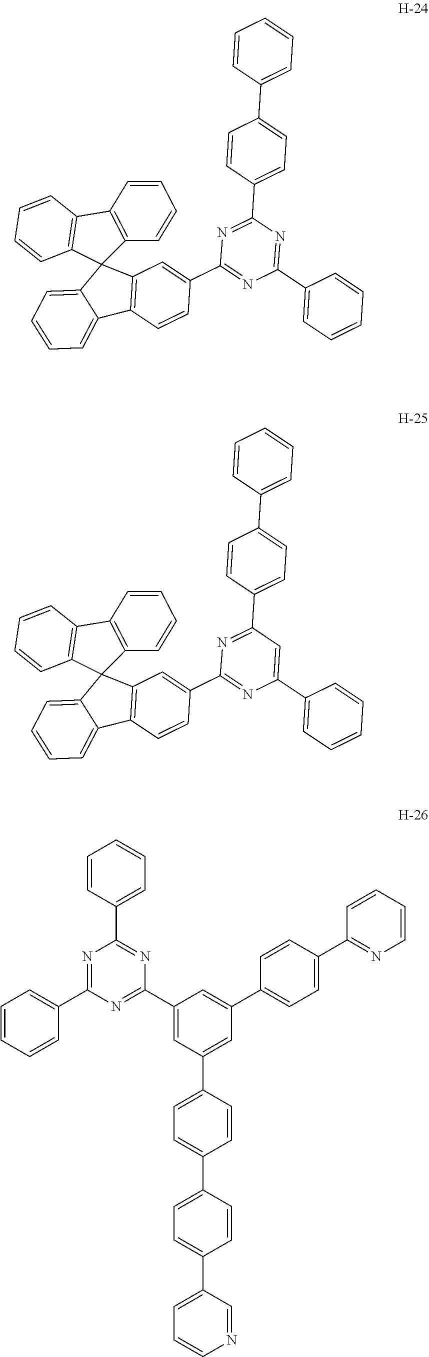 [SMILES: c1ccc(-c2ccc(-c3nc(-c4ccccc4)nc(-c4ccc5c(c4)C4(c6ccccc6-c6ccccc64)c4ccccc4-5)n3)cc2)cc1 ; c1ccc(-c2ccc(-c3cc(-c4ccccc4)nc(-c4ccc5c(c4)C4(c6ccccc6-c6ccccc64)c4ccccc4-5)n3)cc2)cc1 ; c1ccc(-c2nc(-c3ccccc3)nc(-c3cc(-c4ccc(-c5ccc(-c6cccnc6)cc5)cc4)cc(-c4ccc(-c5ccccn5)cc4)c3)n2)cc1]

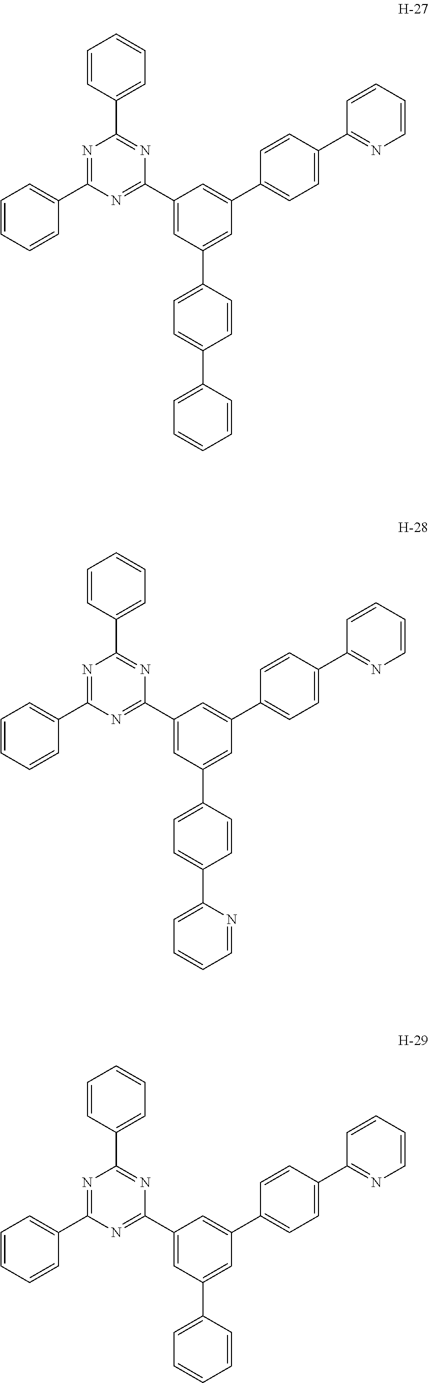 [SMILES: c1ccc(-c2ccc(-c3cc(-c4ccc(-c5ccccn5)cc4)cc(-c4nc(-c5ccccc5)nc(-c5ccccc5)n4)c3)cc2)cc1 ; c1ccc(-c2nc(-c3ccccc3)nc(-c3cc(-c4ccc(-c5ccccn5)cc4)cc(-c4ccc(-c5ccccn5)cc4)c3)n2)cc1 ; c1ccc(-c2cc(-c3ccc(-c4ccccn4)cc3)cc(-c3nc(-c4ccccc4)nc(-c4ccccc4)n3)c2)cc1]

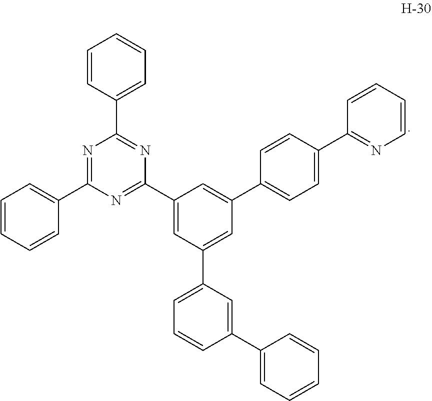 [SMILES: c1ccc(-c2cccc(-c3cc(-c4ccc(-c5ccccn5)cc4)cc(-c4nc(-c5ccccc5)nc(-c5ccccc5)n4)c3)c2)cc1]

11. The organic electroluminescent diode according to claim 1, wherein the HOMO energy level of the host material is greater than −6.55 eV and less than −5.75 eV, and the LUMO energy level of the host material is greater than −3.2 eV and less than −2.4 eV.

12. The organic electroluminescent diode according to claim 1, wherein the absolute value of the energy level difference between the HOMO level of the material of the hole blocking layer and the HOMO level of the host material is not less than 0.15 eV, and the absolute value of the energy level difference between the first triplet energy level of the hole blocking material and the first triplet energy level of the host material is not greater than 0.15 eV.

13. The organic electroluminescent diode according to claim 12, wherein the first triplet energy level of the material of the hole blocking layer is less than the first triplet energy level of the host material, and the absolute value of the energy level difference between the first triplet energy level of the material of the hole blocking layer and the first triplet energy level of the host material is less than 0.1 eV.

14. The organic electroluminescent diode according to claim 1, wherein the electron migration rate of the material of the hole blocking layer is not less than 5×10⁻⁶ cm²/Vs.

15. The organic electroluminescent diode according to claim 1, wherein the absolute value of the energy level difference between the LUMO energy level of the material of the hole blocking layer and the LUMO energy level of the host material is less than 0.4 eV.

16. The organic electroluminescent diode according to claim 1, wherein the thickness of the hole blocking layer is not greater than 10 nm, and the thickness of the light emitting layer ranges from 10 nm to 30 nm.

17. The organic electroluminescent diode according to claim 1, wherein the absolute value of the energy level difference between the LUMO energy level of the material of the electron transport layer and the LUMO energy level of the material of the hole blocking layer is less than 0.5 eV, and the first triplet energy level of the material of the electron transport layer is smaller than the first triplet energy level of the material of the hole blocking layer.

18. The organic electroluminescent diode according to claim 1, wherein the electron migration rate of the electronic transport layer is not more than 100 times of the electron migration rate of the hole blocking layer.

19. The organic electroluminescent diode according to claim 1, wherein the electron transfer layer includes a first electron transfer layer and a second electron transfer layer disposed in layers, the first electron transfer layer is located on the side of the hole blocking layer away from the second electron transfer layer, and the first triplet energy levels of the hole blocking layer, the second electron transport layer and the first electron transport layer sequentially decrease.

20. A display panel, comprising the organic electroluminescent diode, wherein the organic electroluminescent diode comprises an anode, a light emitting layer, a hole blocking layer, an electron transport layer, and a cathode, which are stacked in sequence; wherein the light emitting layer comprises a host material, a TADF material, and a fluorescent dopant material:
the host material is selected from the compound represented by Chemical Formula 1. and the material of the hole blocking layer is selected from the compound represented by Chemical Formula 2: 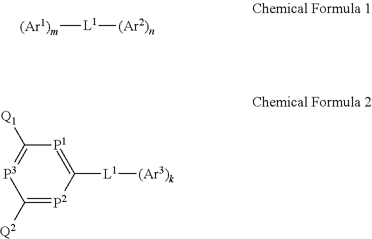
wherein, m and n are the same or different, and are independently selected from an integer not less than 1; K is 1 or 2;
L¹ is selected from a single bond, and substituted or unsubstituted aryl group with 6 to 12 ring-forming carbon atoms. In a case that L¹ has a substituent, the substituent is selected from deuterium, fluorine, cyano, alkyl group with 1 to 4 carbon atoms, deuteroalkyl group with 1 to 4 carbon atoms, fluoroalkyl group with 1 to 4 carbon atoms, and aryl group with 6 to 12 ring-forming carbon atoms;
Ar¹ is selected from the groups shown in Chemical Formulas 1-A. 1-B, and 1-C: 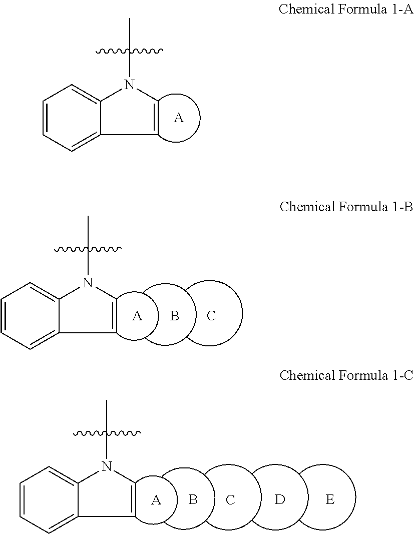
Ring A, Ring C, and Ring E are independently substituted or unsubstituted benzene ring. In a case that Ring A, Ring C, or Ring E has a substituent, the substituent is selected from deuterium, fluorine, cyano, alkyl group with 1 to 4 carbon atoms, deuteroalkyl group with 1 to 4 carbon atoms, and fluoroalkyl group with 1 to 4 carbon atoms;
Ring B is 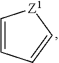
and Ring D is 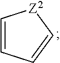
Z¹ and Z² are independently selected from NR¹. O. S. C(R²R³), Si(R²R³), and Ge(R²R³); wherein, R¹, R², and R³ are the same or different, and each independently selected from hydrogen, alkyl group with 1 to 4 carbon atoms, and aryl group with 6 to 12 ring-forming carbon atoms;
Ar² is selected from the groups shown in Chemical Formulas 1-D, 1-E, 1-F, and 1-G: 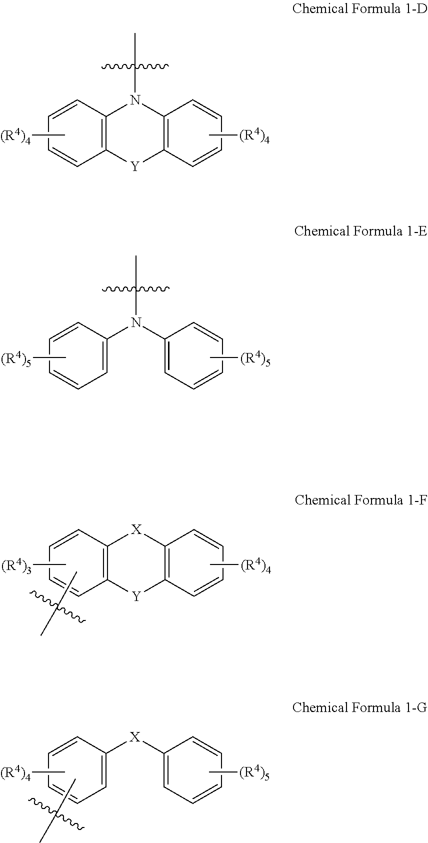
each R⁴ is the same or different, and is independently selected from hydrogen, deuterium, fluorine, cyano, alkyl group with 1 to 4 carbon atoms, deuteroalkyl group with 1 to 4 carbon atoms, fluoroalkyl group with 1 to 4 carbon atoms, aryl group with 6 to 12 ring-forming carbon atoms, and heteroaryl group with 3 to 15 ring-forming carbon atoms;
X and Y are independently selected from NR⁵, O. S. C(R⁶R⁷), Si(R⁶R⁷), Ge(R⁶R⁷). R⁵ R⁶, and R⁷ are independently selected from hydrogen, alkyl group with 1 to 4 carbon atoms, and aryl group with 6 to 12 ring-forming carbon atoms, and Y can also be a single bond;
and Ar¹ and Ar² are not simultaneously N-carbazolyl group;
L² is selected from a single bond, substituted or unsubstituted aryl group with 6 to 12 ring-forming carbon atoms. In a case that the L² has a substituent, the substituent is selected from deuterium, fluorine, cyano, alkyl group with 1 to 4 carbon atoms, deuteroalkyl group with 1 to 4 carbon atoms, and fluoroalkyl group with 1 to 4 carbon atoms;
p¹, p², and P³ are the same or different, each independently selected from N or CH, and at least two are N;
Ar³ is selected from the groups shown in Chemical Formulas 2-A and 2-B: 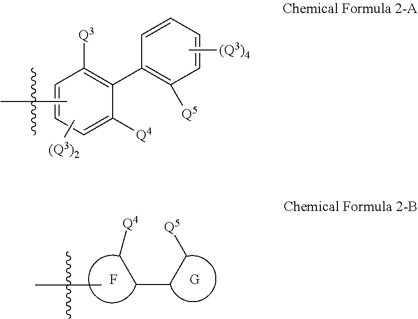
Ring F and Ring G are independently selected from benzene ring or pyridine ring, and at least one is pyridine ring;
Q¹, Q², and each Q³, Q⁴, and Q⁵ are the same or different, and are independently selected from hydrogen, deuterium, cyano, fluorine, substituted or unsubstituted aryl group with 5 to 50 carbon atoms, and substituted or unsubstituted alkyl group with 1 to 50 carbon atoms. Alternatively, Q⁴ and Q⁵ can be condensed to form 5- to 7-membered cycle with connected groups.
